(19) United States
(12) Patent Application Publication
(10) Pub. No.: US 2025/0033048 A1
(43) Pub. Date: Jan. 30, 2025

(54) MICROFLUIDIC DEVICES WITH DEHYDRATED REAGENTS

(71) Applicant: Hewlett-Packard Development Company, L.P., Spring, TX (US)

(72) Inventors: Raghuvir N. Sengupta, Palo Alto, CA (US); Viktor Shkolnikov, Palo Alto, CA (US); Steven Barcelo, Palo Alto, CA (US)

(73) Assignee: Hewlett-Packard Development Company, L.P., Spring, TX (US)

(21) Appl. No.: 18/713,121

(22) PCT Filed: Dec. 15, 2021
(86) PCT No.: PCT/US2021/063596

§ 371 Date: May 23, 2024

(51) Int. Cl. B01L 3/00

(52) U.S. Cl. CPC B01L 3/502738; B01L 2200/14; B01L 2200/16; B01L 2300/0864; B01L 2400/043; B01L 2400/0442; B01L 2400/0677; B01L 2400/0683

(57) ABSTRACT

An example microfluidic device comprises a plurality of fluid channels, an unblocking actuator, and a fluid ejection device fluidically coupled to the plurality of fluid channels. The plurality of fluid channels include a plurality of dehydrated reagents disposed within the plurality of fluid channels and a plurality of fluid blocking regions including blocking material disposed between the first fluid chamber and the plurality of dehydrated reagents. The unblocking actuator is coupled to the plurality of fluid channels proximal to the plurality of fluid blocking region.

### BACKGROUND

Microfluidic devices may be used to perform operations on fluids, such as the manipulation of fluid droplets to facilitate the handling and testing of fluids on a small scale. Such devices may be used in the medical industry, for example to analyze antibiotic susceptibility, analyze deoxyribonucleic acid (DNA), detect pathogens, perform clinical diagnostic testing, and/or for synthetic chemistry, among other types of industries and/or purposes.

### BRIEF DESCRIPTION OF THE DRAWINGS

FIGS. 1A-1B illustrate example microfluidic devices with dehydrated reagents, in accordance with the present disclosure.

FIG. 2 illustrates an example reservoir, in accordance with the present disclosure.

FIGS. 3A-3K illustrate further example microfluidic devices with dehydrated reagents, in accordance with the present disclosure.

FIG. 4 illustrates an example operation of a microfluidic device with dehydrated reagents, in accordance with the present disclosure.

FIGS. 5A-5B illustrate another example microfluidic device with dehydrated reagents and a plurality of dedicated fluid ejection devices, in accordance with the present disclosure.

FIG. 6 illustrates an example device including non-transitory computer-readable storage medium, in accordance with examples of the present disclosure.

FIG. 7 illustrates an example method for ejecting reconstituted dehydrated reagent from a microfluidic device, in accordance with examples of the present disclosure.

FIGS. 8A-8D illustrate different example systems including a fluid dispensing device and a first microfluidic device with dehydrated reagents, in accordance with the present disclosure.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense, and the scope of the present disclosure is defined by the appended claims. It is to be understood that features of the various examples described herein may be combined, in part or whole, with each other, unless specifically noted otherwise.

Different types of tests and/or chemical reactions may involve manual handling and manipulation of reagents and/or sample fluid being tested by a user, such as pipetting fluids to a substrate. Manual handling may be burdensome and may reduce efficiencies in performing the operations and increase risk for errors. In some instances, microfluidic devices may be used to perform operations on fluids, which may reduce manual handling of fluid components. The microfluidic device may be a cartridge which is loaded with different fluids, and inserted or disposed in a fluid dispending device to selectively eject fluids to a substrate. For some tests, and even with the use of microfluidic devices, reagents may be manually handled and/or manipulated. For example, for antibiotic susceptibility test, such as broth dilution assays and Epsilometer test (E-test), bacterial inoculum may be added to regions of substrate with different antibiotics, which are inoculated together and assessed optically to evaluate the amount of bacterium remaining. In some instances, a single antibiotic of a particular concentration is tested per region of the substrate. For antibiotic susceptibility tests and other types of tests, different combinations of reagents and concentrations may be tested, with microfluidic devices being used to increase testing volume and minimize user handling. The microfluidic device may be loaded with different reagents and used to eject different combinations and concentrations of the reagents. However, liquid reagents may degrade over time, presenting issues for transporting and storing the microfluidic device.

Examples in accordance with the present disclosure are directed to microfluidic devices, instructions and methods for controlling microfluidic devices, and systems including a microfluidic device which includes a plurality of dehydrated reagents disposed within fluid channels. By incorporating dehydrated reagents, the microfluidic devices may have longer shelf-lives and are easier to transport and store, e.g., less restriction on temperature, compared to microfluidic devices with fluid reagents. The plurality of fluid channels include blocking regions with blocking material that block fluid access to the dehydrated reagents. The blocking material may be upstream from the dehydrated reagents and block or prevent the flow of fluid from reaching the dehydrated reagents. The flow of fluid may be selectively unblocked to provide fluid to a dehydrated reagent and reconstitute the dehydrated reagent in the fluid. The reconstituted dehydrated reagent may be flown to a fluid ejection device and ejected to a substrate external from the microfluidic device. The fluid channels may each include a different reagent, which may be used to eject a particular reagent and/or a combination of reagents.

As used herein, a dehydrated reagent includes and/or refers to a dried or lyophilized reagent which is disposed in a fluid channel. Reagent lyophilization involves dehydration of frozen reagent aliquots by sublimation under an applied vacuum. A reconstituted dehydrated reagent includes and/or refers to a reagent which is rehydrated and reconstituted in the fluid, which may be referred to as a “rehydrated reagent” and/or is otherwise no longer dehydrated. A fluid channel includes and/or refers to a path through which a fluid or semi-fluid may pass, which may allow for transport of volumes of fluid on the order of microliters, nanoliters, picoliters, or femtoliters.

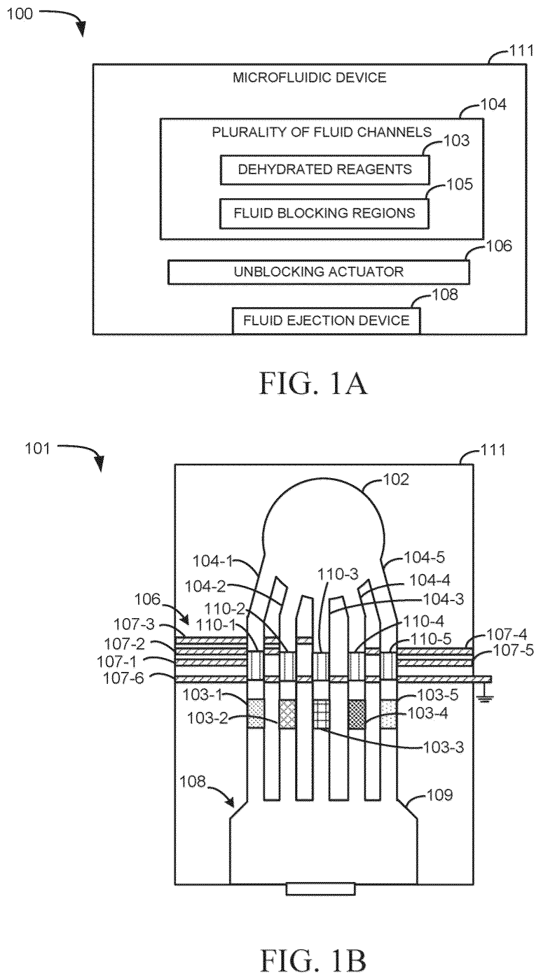

FIGS. 1A-1B illustrate example microfluidic devices with dehydrated reagents, in accordance with the present disclosure. Microfluidic devices, including the microfluidic devices 100, 101 of FIGS. 1A-1B, may be disposable devices used to perform different operations on fluid flown therein, and which may be inserted into and/or disposed within a fluid dispensing device, such as a fluid inkjet device for ejecting fluids to external substrates.

As shown by FIG. 1A, the microfluidic device 100 includes a plurality of fluid channels 104, an unblocking actuator 106, and a fluid ejection device 108. Each of the components of the microfluidic device 100 may be formed on or coupled to a substrate 111. The substrate 111 may comprise a silicon based wafer or other similar materials used for microfabricated devices (e.g., glass, gallium arsenide, plastics, etc.). In some examples, the microfluidic device 100 may include a housing, such as a cover or lid over the substrate 111. As further described herein, examples may comprise fluid channels 104 and/or chambers. Fluid channels 104 and/or chambers may be formed by etching or micromachining processes in the substrate 111. Accordingly, the fluid channels 104 and/or chambers may be defined by surfaces fabricated in the substrate 111 of the microfluidic device 100.

The plurality of fluid channels 104 may be used to flow fluid within the microfluidic device 100 along a fluid path to the fluid ejection device 108. The fluid channels 104 include a plurality of dehydrated reagents 103 disposed within the plurality of fluid channels 104 and a plurality of fluid blocking regions 105. The plurality of fluid blocking regions 105 include blocking material disposed within the plurality of fluid channels 104. Fluid blocking regions 105 include and/or refer to regions of the fluid channels 104 containing the blocking material. The plurality of fluid blocking regions 105 may be upstream from the dehydrated reagents 103, such as being between the dehydrated reagents 103 and a source of fluid.

The plurality of dehydrated reagents 103 may include a variety of different types of reagents, and which may depend on the test to be performed. Non-limiting example dehydrated reagents include an antibiotic, an enzyme, a nucleotide, an antibody, a metabolic indicator, a detectable label, and combinations thereof. In some examples, the microfluidic device 100 includes at least two different reagents, such as different antibiotics, different antibodies, different enzymes, etc., and/or different concentrations of a reagent. Reagents, as used herein, include and/or refer to a substance for use in a biochemical analysis or reaction. In some examples, the plurality of dehydrated reagents 103 include at least two different reagents, at least three different reagents, at least four different reagents, at least five different reagents, or more, such as between two different reagents to ten (or more) different reagents.

Blocking material, as used herein, includes any material which is disposed within a fluid channel and which causes blocking of the flow of fluid. In some examples, the blocking material, along with the unblocking actuator 106, may form a valve that blocks flow of fluid in a closed state and allows fluid flow in an open state. The blocking material may be in a variety of states or forms, such as a solid, liquid, and/or a gas state. Non-limited examples of blocking material include wax, polymer, metal, glass, a magnetic material, ferrofluid, plastic, solid-gas generating material, gas, pressurized gas, and combinations thereof. In some examples, the blocking material is removable from the fluid blocking regions 105. For example, the blocking material may at least partially transition from a solid to a fluid state, such as wax, metal, polymer and other material that may melt in response to heat and/or a solid-gas generating material which may transition to gas in response to heat. In some examples, the blocking material is or forms a permanent component or structure of the microfluidic device 100. For example, the blocking material may form or include valves which are in different states to block or allow fluid flow and/or capillary chambers which may block the flow of fluid, such as by disrupting capillary forces, as further described herein.

The unblocking actuator 106 includes and/or refers to circuitry and/or a physical structure that causes movement of the blocking material and unblocking of the fluid blocking regions 105, such as by removing the blocking material and/or transitioning the blocking material to a different state. In some examples, the unblocking actuator 106 causes the movement in response to an electrical signal provided thereto and/or a source of energy. For example, the unblocking actuator 106 may apply energy to the fluid blocking regions 105 to selectively unblock fluid channels 104. The energy may include heat, pneumatic signals, current or voltage, optical signals (e.g., light), among other energy sources. Example unblocking actuators include a plurality of control lines, a pneumatic source, a plurality of vents, a plurality of permanent magnets or electromagnets, and a plurality of fluid actuators, as well as combinations thereof. The unblocking actuator 106 may form part of an unblocking mechanism used to selectively unblock respective the fluid channels 104.

In some examples, the unblocking actuator 106 includes a plurality of control lines. The plurality of control lines are coupled to the plurality of fluid channels 104 proximal to the plurality of fluid blocking regions 105. In some examples, the plurality of control lines may provide heat to the plurality of fluid blocking regions 105 or may provide electrical signals to other structures forming part of the unblocking mechanism. That is, the control lines may provide electrical signals to the fluid blocking regions 105 and/or to the blocking material. The electrical signals may provide heat to the blocking material and/or cause the blocking material or another component coupled or proximal to the fluid blocking regions 105 to transition states or to otherwise cause unblocking of the fluid blocking regions 105.

In some examples, the blocking material of the plurality of fluid blocking regions 105 is temporarily disposed within the plurality of fluid channels 104, and is removed via application of the electrical signal selectively applied thereto via the plurality of control lines. For example, the plurality of control lines may include a plurality of resistor circuits (e.g., resistor trace lines) disposed to provide heat proximal to the plurality of fluid blocking regions 105 to selectively melt the blocking material. In response to the selectively applied electrical signal, the flow of the fluid is unblocked from an associated fluid channel of the plurality of fluid channels 104 and a respective dehydrated material of the plurality of dehydrated reagents 103 is reconstituted in the fluid and directed to the fluid ejection device 108.

In some examples, the blocking material is in a first state in which fluid flow is blocked and in a second state in which the fluid flow is unblocked. For example, the blocking material may be a solid-gas generating material that is in a solid state and is chemically reacted to a fluid or gas state, as further described below. In other examples, the blocking material may be a metal membrane that disintegrates in response to electrical signals applied thereto.

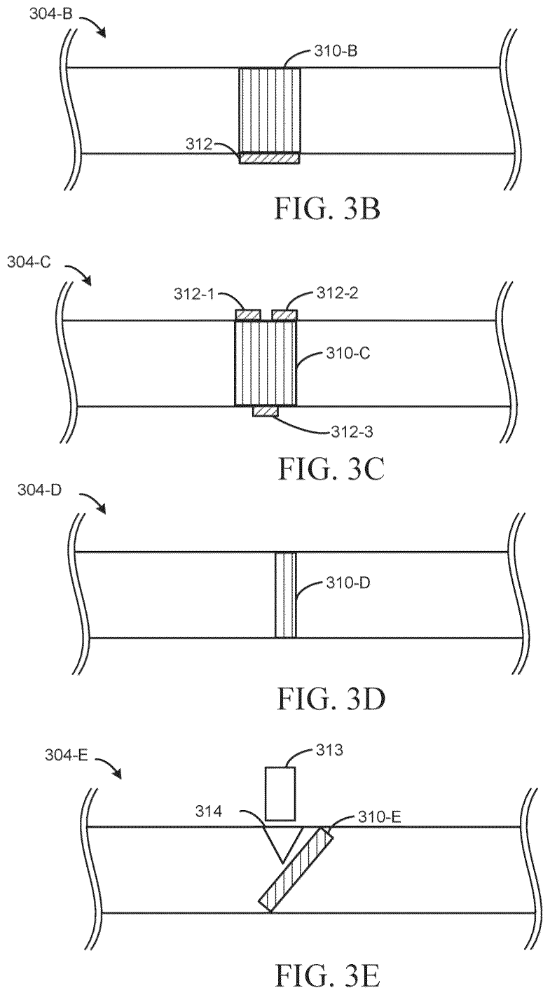

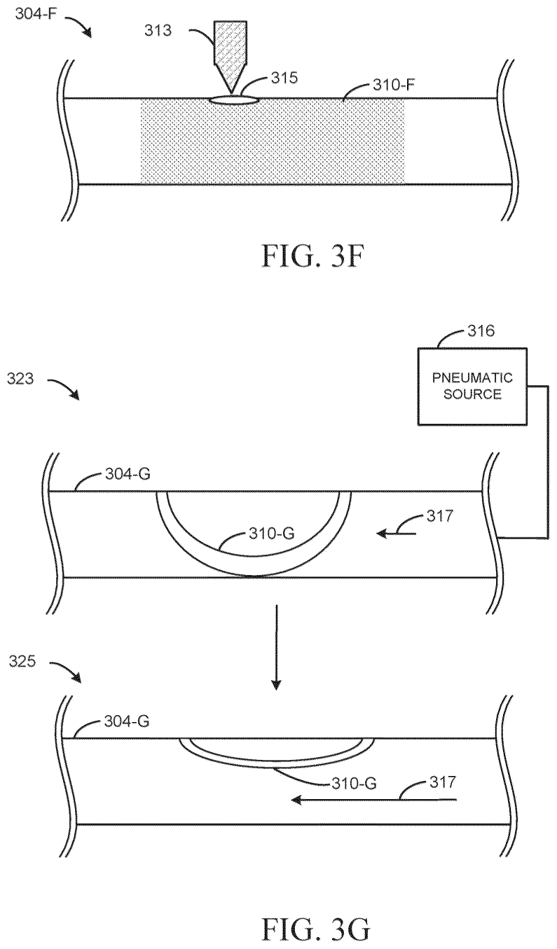

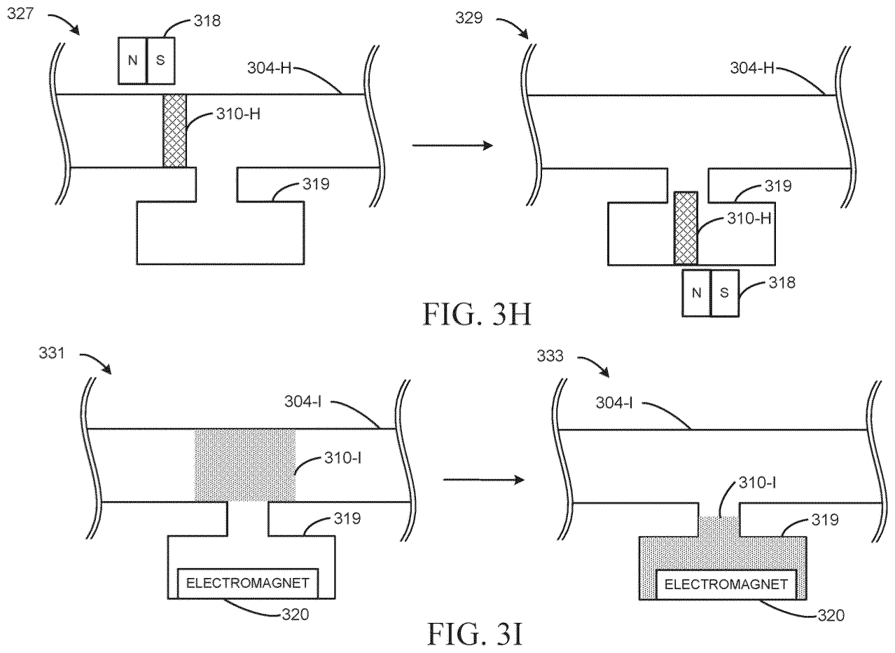

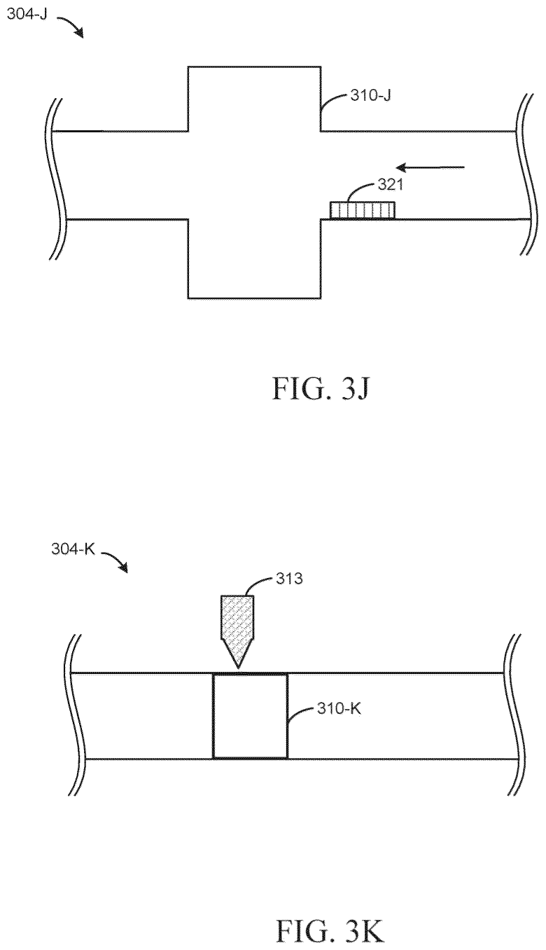

As further described herein and illustrated by FIGS. 3B-3K, the blocking material and unblocking actuator 106 may include a variety of different structures and are not limited to control lines and/or removable blocking material. For example, the blocking material may be or form a permanent component of the microfluidic device 100. In some examples, the unblocking actuator 106 includes a valve or a vent disposed within each of the plurality of fluid blocking regions 105 which is coupled to one of the plurality of control lines. In other examples, the unblocking actuator 106 includes a pneumatic source that provides a pneumatic signal to select ones of the plurality of fluid blocking regions 105. In some examples, the unblocking actuator 106 includes a plurality of magnets disposed proximal to the plurality of fluid blocking regions 105 to provide a magnetic field, which causes magnetic material to move and unblock select ones of the plurality of fluid blocking regions 105. In some examples, the unblocking actuator 106 includes a plurality of fluid actuators disposed proximal to the plurality of fluid blocking regions 105, such as being proximal to a capillary chamber associated with the fluid blocking regions 105. The fluid actuators are actuated to cause flow of fluid.

In any of the above examples, once a fluid blocking region is unblocked, fluid is allowed to flow through an associated fluid channel and a dehydrated reagent of the plurality 103 may be reconstituted in the fluid and directed along the fluid channel to the fluid ejection device 108. The fluid ejection device 108 is fluidically coupled to the plurality of fluid channels 104. The fluid ejection device 108 includes an ejection chamber with a fluid actuator and a nozzle to eject fluid from the microfluidic device 100. A fluid actuator, as used herein, includes and/or refers to circuitry and/or a physical structure that causes movement of fluid. Example fluid actuators include an integrated inertial pump, a thermal inkjet (TIJ) resistor, a piezoelectric device, a magnetostrictive element, an electrode, an ultrasound source, mechanical/impact driven membrane actuators, magneto-restrictive drive actuators, and other suitable components.

Fluid may flow into the fluid ejection device 108 from the plurality of fluid blocking regions 105. The fluid may be from a fluid source, such as a coupled reservoir, and may include any type of fluid which may rehydrate and/or reconstitute the dehydrated reagents 103. The fluid may include a buffer fluid and/or salts, in some examples. The fluid flown to the fluid ejection device 108 may include reconstituted dehydrated reagents from respective ones of the plurality of fluid channels 104 having the blocking material removed, moved, and/or transitioned to an open state. The fluid flows to the respective dehydrated reagents and reconstitutes the dehydrated reagents within the fluid, and the fluid with the reconstituted dehydrated reagents is flown to the fluid ejection device 108. The fluid actuator of the fluid ejection device 108 may be actuated to cause flow of fluid within the ejection chamber and ejection of a volume of the fluid from the fluid ejection device 108.

In some examples, fluid with a first reconstituted dehydrated reagent is mixed with fluid with a second or more reconstituted dehydrated reagent to provide a mixture of at least two reconstituted dehydrated reagents. In other examples, a single reconstituted reagent is ejected from the microfluidic device 100 via the fluid ejection device 108.

As described above, the fluid ejection device 108 includes an ejection chamber coupled to a nozzle, and with a fluid actuator disposed in the ejection chamber. The nozzle may include an orifice used for ejecting fluid from the ejection chamber. The fluid ejection device 108 may include a drop-on-demand thermal bubble system including a TIJ ejector. The TIJ ejector may implement a thermal resistor in the ejection chamber (which is coupled to the illustrated second fluid chamber 109 of FIG. 1B) and create bubbles that force fluid drops out of the nozzle. In some examples, the fluid may be ejected from the microfluidic device 100 by the fluid ejection device 108 that includes a drop-on-demand piezoelectric inkjet system including a piezoelectric inkjet (PIJ) ejector that implements a piezoelectric material actuator as an ejection element to generate pressure pulses that force fluid drops out of the nozzle. Examples are not so limited and additional and/or different types of fluid ejection device 108 may be used to eject fluid from the ejection chamber. Similarly, different and/or additional components may be coupled to the microfluidic device 100 to eject fluid therefrom, such as a fluid dispensing device and other components of a system for driving biochemical reactions.

In various examples, the microfluidic device 100 includes additional components, such as chambers, actuators, fluid reservoirs, and other components. For example, and as further illustrated and described by FIG. 1B, the microfluidic device 100 may further include a reservoir containing a fluid and a fluid chamber coupled to the reservoir and the plurality of fluid channels 104. In some examples, the microfluidic device 100 may include additional fluid ejection devices, such as fluid ejection devices which are coupled to and/or dedicated to a particular fluid channel of the plurality of fluid channels 104.

In some examples, the microfluidic device 100 may further include a fluid control actuator. A fluid control actuator, as used herein, includes and/or refers to circuitry and/or a physical structure that causes activation of fluid flow within the microfluidic device. Example fluid control actuators include a plunger, unblocking actuator 106, a fluid actuator, and combinations thereof. For example, the fluid flow may be provided by a fluid actuator that include a thermal resistor, such as a TIJ resistor. The resistor may be activated to create a vapor bubble within a fluid channel and/or chamber that disperses fluid. Energy may be applied to the resistor that super heats the resistor and a volume of surrounding fluid. The energy is removed from the resistor, which causes the vapor bubble to collapse. During vapor bubble collapse, a fluidic bubble jet may be produced that concentrates the residual kinetic energy of the bubble to provide a high pressure. In some examples, the vapor bubble may cause movement of fluid along a fluid channel and/or the plurality of fluid channels 104. However, examples are not so limited and fluid flow may be provided in a variety of ways, including but not limited to, fluid pumps, electrodes providing electrowetting forces or ions, magnetic sources, and gravity, among others.

FIG. 1B illustrates an example microfluidic device 101. The microfluidic device 101 of FIG. 1B may include an implementation of and/or include similar features and components as the microfluidic device 100 of FIG. 1A, with further detail illustrated. For instance, the microfluidic device 101 includes a plurality of fluid channels 104-1, 104-2, 104-3, 104-4, 104-5 (herein generally referred to as “the plurality of fluid channels 104” for ease of reference) having a plurality of fluid blocking regions and a plurality of dehydrated reagents 103-1, 103-2, 103-3, 103-4, 103-5 (herein generally referred to as “the plurality of dehydrated reagents 103” for ease of reference), an unblocking actuator 106 which includes a plurality of control lines 107-1, 107-2, 107-3, 107-3, 107-4, 107-5, 107-6 (herein generally referred to as “the plurality of control lines 107” for ease of reference) and the fluid ejection device 108.

As shown by FIG. 1B, the plurality of fluid blocking regions include a plurality of blocking material 110-1, 110-2, 110-3, 110-4, 110-5 disposed within the plurality of fluid channels 104. The blocking material 110-1, 110-2, 110-3, 110-4, 110-5 may be disposed between a fluid source (e.g., the first fluid chamber 102) and the plurality of dehydrated reagents 103 and may block fluid from reaching the dehydrated reagents 103, such as blocking the flow of fluid along the plurality of fluid channels 104. In some examples, each fluid blocking region of a fluid channel includes a different dehydrated reagent from the remaining dehydrated reagents, such as a different type of reagent and/or different concentration of a reagent.

In some examples, a different control line of the plurality of control lines 107 is coupled to one of the respective fluid blocking regions proximal to a respective blocking material 110 of the microfluidic device 101. As previously described, electrical signals may be applied to the control lines 107 to selectively unblock a select fluid channel of the plurality of fluid channels 104.

In some examples, and as illustrated by FIG. 1B, the blocking material may include material that melts in response to heat and/or that transitions to a gas state in response to heat, such as solid-gas generating material. Example materials include wax, metal, and a solid-gas generating material.

A solid-gas generating material, as used herein, includes and/or refers to a material that is chemically reactive to form a gas. In some examples, the solid gas-generating material is chemically reactive to form a gas by a thermal decomposition reaction, by a combustion reaction, or by a chemical reaction with a fluid in the fluid channel. Example solid gas-generating material include an Azobis compound, a peroxide, a carbonate, a nitrate, a nitrite, an azide, nitrocellulose, and a combination thereof. In some examples, the microfluidic device 101 further includes a solid gas-absorbing material in each of the fluid channels 104 adjacent to the solid gas-generating material. A solid gas-absorbing material is a material that absorbs gas. In other examples, the fluid channels 104 may include a gas vent downstream or upstream of the solid gas-generating material to allow the gas to escape from the microfluidic device 101. Thermal heat, such as from the control lines 107, resistors, and/or a spark plug, may initiate the chemical reaction that converts the solid gas-generating material to gas, which may be released from the microfluidic device 101 via a vent and/or the fluid channel itself and which allows for fluid to flow. A spark plug may include two electrodes separated by a gap. When a sufficient voltage difference is applied between the two electrodes, a spark or arc may form between the electrodes. This spark may ignite the solid gas-generating material.

In some examples, the plurality of control lines 107 are disposed to provide heat proximal to the plurality of fluid blocking regions to selectively melt and/or react to the blocking material 110. For example, the control lines 107 may be a plurality of resistors circuits (e.g., resistor trace lines) having electrical signals are applied thereto and which carry the electrical signals as current and/or a voltage. In some examples, the blocking material 110 may have a melting point of around 100 degrees Celsius (C) or less and/or is inert.

However, examples are not limited to applying heat to the fluid blocking regions and melting or transitioning the blocking material 110 to fluid and/or gas. In some examples, the electrical signals are carried along the plurality of control lines 107 to another component proximal to the blocking material 110, as further described and illustrated by FIGS. 3B-3K and which includes vents, pneumatic valves and other types of valves, capillary chambers, among other variations. In any of the various examples described herein, the blocking material 110 may include or function as a valve positioned across the fluid blocking region, and which is in a closed state and blocks the flow of fluid. The electrical signal may be provided by a coupled control line of the plurality of control lines 107, and which causes the valve to transition from the closed state to the open state such that fluid may flow through the fluid blocking region and to the dehydrated reagent downstream from the fluid blocking region.

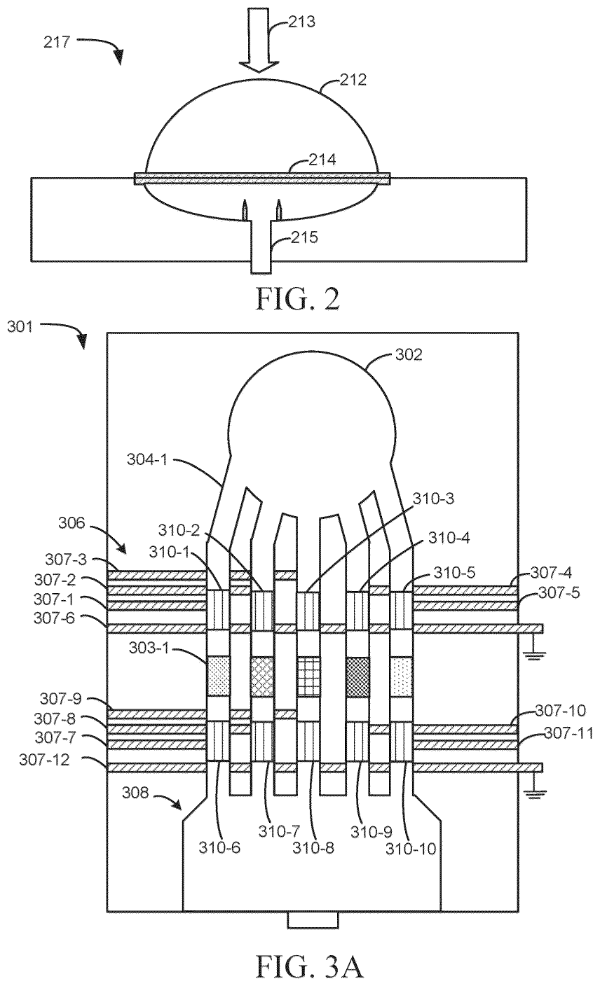

In various examples, the microfluidic device 101 further includes a first fluid chamber 102 coupled to a reservoir that contains a fluid and is fluidically coupled to the plurality of fluid channels 104. As used herein, a chamber includes and/or refers to an enclosed and/or semi-enclosed region of the device, which is capable of storing fluid. In some examples, the reservoir may form part of the microfluidic device 101 and, in other examples, may be a separate component. A reservoir is a type of chamber that stores a fluid for inputting to the microfluidic device 101 and/or fluid channels 104. An example reservoir is illustrated by FIG. 2.

The fluid ejection device 108 may include or be formed on a die and may include a second fluid chamber 109 which forms part of the fluid ejection device 108. For example, the second fluid chamber 109 may form part of the package of the die. In other examples, the second fluid chamber 109 may be separate from and fluidically coupled to the fluid ejection device 108. In some examples, the second fluid chamber 109 is fluidically coupled to and/or shared by each of the plurality of fluid channels 104. Fluid from the channels 104, including reconstituted dehydrated reagents, may mix in the second fluid chamber 109 prior to or in response to being ejected from the microfluidic device 101.

In some examples, the second fluid chamber 109 may include beads and/or a fluid actuator to mix fluids within. In other examples, the beads may be located on a substrate that the fluid is ejected to, as further described herein. As used herein, a bead refers to and/or includes a material formed in a three-dimensional shape, such as a sphere, an ellipsoid, oblate spheroid, and prolate spheroid shapes. The beads may be formed of a variety of different materials, such as polymer, glass, silica, silicon carbide, tungsten carbide iron oxide steel, silica coated metal, ion oxide, a soft ferrite, a ferromagnetic material, a ferrimagnetic material, and/or boron nitride, among other material and combinations thereof. In some examples, a fluid actuator may be located in the second fluid chamber 109 and may be activated to actuate and cause mixture (e.g., stirring) of the fluid therein.

The fluid is flowed to the fluid ejection device 108 and ejected to a substrate containing a sample fluid. The sample fluid, as used herein, includes and/or refers to a fluid containing a component to be tested, such as a bacterial inoculum, fluid from a subject (e.g., spit, blood or plasma, urine or other fluids), among other types of biological fluids with components to be tested. A biochemical reaction may occur between the fluid, the reconstituted dehydrated reagent(s), and the sample fluid. The fluid stored by the reservoir may include the buffer fluid used to assist and/or drive the biochemical reaction.

As an example, for an antibiotic susceptibility test, the reagents may include different antibiotics and a metabolic indicator. A metabolic indicator includes and/or refers to a molecule or compound that transforms to an optical indicator in the presence of particular cells, such as molecules that enzymes act upon. An optical indicator includes and/or refers to a molecule or compound that is optically detectable, such as a fluorescent molecule or compound. The optical indicator may be detected visually (e.g., by the human eye) or using a detector, such as via colorimetric, fluorescence, or other luminescence detection, such as Raman. The metabolic indicator may be ejected to the plurality of regions of the substrate concurrently with a reconstituted antibiotic and/or prior to or after. For example, the fluid channel containing the metabolic indicator may be unblocked by removing the respective blocking material and/or transitioning states of the blocking material, and in response the metabolic indicator is ejected to each of the plurality of regions of the substrate. The remaining fluid channels may contain dehydrated antibiotics. The dehydrated antibiotics may be ejected in different concentrations to respective ones of the plurality regions of the substrate, such that each region of the substrate contains the metabolic indicator and either a different antibiotic and/or different concentrations of an antibiotic. The bacterium inoculum may already be disposed within the regions of the substrate, either by ejection by another microfluidic device or is pre-deposited, and/or may subsequently be provided thereto. A subset of the regions of the substrate may include controls, which have the bacterium inoculum and the metabolic indicator and no antibiotic. The remaining regions may be split into subgroups with each subgroup including different concentrations of a respective antibiotic as well as the bacterium inoculum and the metabolic indicator. In other examples and/or in addition, mixtures of the antibiotics may be tested.

The components within the substrate regions are incubated by applying heat to the substrate to allow for the bacteria to grow, such as applying heat of around 37 degrees C. If the bacteria grows, e.g., is present, the metabolic indicator undergoes a chemical transformation to form a fluorescent compound which may be detected. With more bacteria, additional fluorescent compound may be present within the regions of the substrate, resulting in a stronger fluorescent signal. As the bacteria concentration increases (e.g., the antibiotic does not kill the bacteria), the metabolic indicator is transformed to the fluorescent compound and results in an increase in fluorescence signal until no metabolic indicator remains and the florescence signal drops. In some examples, the regions of the substrate may be observed over time to observe both the peak fluorescent signal and the time or speed of the increase in fluorescent signal. The antibiotics may be individually analyzed and/or then used to identify combinations and/or mixtures for further testing.

Examples are not limited to antibiotics used as reagents, and the above may be applied to different types of tests. For example, the reagents may include pharmaceutical drugs, with the sample fluid including fluid from a subject containing cancer cells. The pharmaceutical drugs may be tested to detect impact on cellular growth, such as the impact on the growth of cancer cells and/or to healthy cells. The metabolic indicator may be transformed by the cancer cells or the healthy cells, and the cellular growth is observed via the change in fluorescent signals.

As another example, the reagents may include an antibody or antigen, which is used to test for a target in a sample that binds to the antibody or antigen. The binding may be optically detected by the metabolic indicator or another detectable label. Examples are not limited to antibody-antigen detections, and may include any type of affinity probes, such as aptamers.

Different example metabolic indicators may be used. One example is resazurin which is transformed to resorufin when mixed with living cells, such as with bacteria. When mixed with cells, resazurin diffuses into the cell where it is irreversibly reduced to resorufin within the cell. The resorufin may further reversibly reduced to dihydroresorufin. Resorufin is fluorescent, which allows for monitoring the reaction. For example, resorufin is excited at around 540 nanometers (nm) and emits around 590 nm. Living cells, such as bacteria, may be mixed with resazurin for around one to four hours. For example, after incubation and to overcome the background signal, around 7×106 colony forming unit per milliliter (cfu/mL) of Escherichia coli may be present.

Examples are not limited to resorufin, and other types of metabolic indicators or detectable labels may be used. Other example metabolic indicators include nitrophenol, 4-methylumbelliferone (4-MU), 7-amin-4-methylcoumarin (7-AMC), 7-hydrocycoumarin-3-carboxylate (EHC), fluorescein, dihydroxynapthalenes, indoxyl, Aldols™, and ELF™. In some examples, a metabolic indicator may not be used. For example, a reagent may be labeled with a detectable label, such as optically (e.g., attached fluorophore) or electrically labeled antibody or enzyme. In other examples, the biochemical reaction may be observed and/or analyzed without any labels and/or metabolic indicators. A detectable label, as used herein, includes and/or refers to a compound or molecule that may be detected optically, electrically, or otherwise.

As noted above, the microfluidic devices 100, 101 may be coupled to circuitry to control the flow of fluid and the reagents from the microfluidic device 100, 101. For instance, a processor forming part of a device may be coupled to the plurality of control lines 107 to control electrical signals (e.g., heat) applied to the fluid blocking regions and/or other components associated with the fluid blocking regions to unblock the flow of fluid.

FIG. 2 illustrates an example reservoir, in accordance with the present disclosure. The reservoir 217 may form part of the microfluidic device and/or be coupled thereto.

In some examples, the reservoir 217 include a blister pack. For example, the reservoir 217 may include a reservoir portion 212 containing the fluid, sometimes referred to as a “blister”, with a layer of breakable material 214 coupled to the reservoir portion 212. The reservoir portion 212 includes a chamber which is formed of a flexible material. Breakable material, as used herein, includes and/or refers to material which may be pierced, torn, or otherwise broken. The breakable material 214 may include aluminum foil, plastic, and other types of materials which may be pierced and/or otherwise break. Prior to breaking the layer of breakable material 214, fluid may be contained within the reservoir portion 212 and the reservoir 217 may be coupled to an inlet of the first fluid chamber (e.g., chamber 102 of FIG. 1B) of a microfluidic device.

A force may be applied to the layer of breakable material 214 to cause the reservoir 217 to fluidically couple to the first fluid chamber and/or the plurality of fluid channels coupled to an outlet of the first fluid chamber. For example, the reservoir portion 212 may be formed of a flexible material, such that a force 213 (e.g., pressing) on the flexible material causes pressure on the layer of breakable material 214 via the fluid filled therein and cause the layer of breakable material 214 to break. In some examples, piercing structures may be located below the layer of breakable material 214 to assist with breaking the breakable material 214. In response to the break, fluid in the reservoir portion 212 flows to a fluid channel 215 that is coupled to the first fluid chamber and/or the plurality of fluid channels of the microfluidic device. A piercing structure, as used herein, includes and/or refers to an object with a sharp point or edge.

In other examples, the reservoir 217 is under pressure. In such examples, the force 213 applied to the reservoir 217 may be caused by removal of blocking material and/or changing a state of the blocking material, which unblocks a respective fluid channel. The unblocking of the fluid channel may change the pressure of the microfluidic device, which causes the force applied to the reservoir portion 213 (e.g., pulling on the layer of breakable material 214 which causes the break).

Examples are not limited to blister packs and may include other types of reservoirs and a fluid control actuator. For example, the fluid may be contained in a reservoir coupled to the first fluid chamber and/or plurality of fluid channels, in which fluid from the reservoir is prevented by a valve and/or otherwise does not occur until activated, such as via a fluid actuator.

FIGS. 3A-3K illustrate other example microfluidic devices with dehydrated reagents, in accordance with the present disclosure.

The microfluidic device 301 of FIG. 3A include similar features and components as the microfluidic devices of FIG. 1B, with additional blocking regions and control lines, and is numbered accordingly. For instance, the microfluidic device 301 of FIG. 3A includes a plurality of fluid channels (as illustrated by the labeled fluid channel 304-1) having a plurality of fluid blocking regions and a plurality of dehydrated reagents (as illustrated by the labeled dehydrated reagent 303-1), an unblocking actuator 306 including the plurality of control lines 307-1, 307-2, 307-3, 307-4, 307-5, 307-6, 307-7, 307-8, 307-9, 307-10, 307-11, 307-12 (herein generally referred to as “the plurality of control lines 307” for ease of reference) and the fluid ejection device 308.

In some examples, the microfluidic device 301 includes the plurality of fluid blocking regions including the blocking material 310-1, 310-2, 310-3, 310-4, 310-5 disposed within the plurality of fluid channels between the first fluid chamber 302 and the plurality of dehydrated reagents (as illustrated by 303-1), and additional blocking material 310-6, 310-7, 310-8, 310-9, 310-10 disposed between the plurality of dehydrated reagents and the fluid ejection device 308. The stability of the dehydrated reagents may decrease in response to moisture exposure. The additional blocking material 310-6, 310-7, 310-8, 310-9, 310-10 may mitigate or reduce exposure of the dehydrated reagents to moisture, and may increase the shelf life of the microfluidic device 301. The dehydrated reagents are disposed between blocking material (e.g., between the blocking material 310-1, 310-2, 310-3, 310-4, 310-5 and the additional blocking material 310-6, 310-7, 310-8, 310-9, 310-10), which may minimize risk of moisture-induced degradation of the dehydrated reagent.

As further illustrated, the control lines 307 are respectively coupled to each of the fluid blocking regions proximal to each of the blocking material 310-1, 310-2, 310-3, 310-4, 310-5, 310-6, 310-7, 310-8, 310-9, 310-10, which may operate as previously described. The control lines 307 may be disposed beneath the fluid channels and thermal coupled to a respective fluid blocking region. To unblock a respective fluid channel, fluid blocking material in both fluid blocking regions of the respective fluid channel, such as blocking material 310-1 and 310-6 of fluid channel 304-1, may be removed and/or transition states.

As described above, examples are not limited to blocking material which includes removable material, such as wax or solid-gas generating material, and/or an unblocking actuator that includes control lines which include apply heat. FIGS. 3B-3K show different variations of blocking material and/or unblocking actuators, which may be used in different microfluidic devices. While FIGS. 3B-3K show a close up view of the plurality of fluid blocking regions and/or a fluid blocking region, the microfluidic device may include substantially the same features as previously described herein, including the plurality of fluid channels and the fluid ejection device.

FIGS. 3B-3D, as well as FIG. 3F, illustrate example blocking material 310-B, 310-C, 310-D, 310-F that includes removable material which melts or transitions states in response to energy applied thereto. FIG. 3B illustrates blocking material 310-B in a fluid channel 304-B which is heated using a heat source 312. The heat source 312 may include a control line that is thermally coupled to the fluid channel 304-B. In some examples, the control line may provide an electrical signal to another heat source, such as a resistor coupled to the control line. In various examples, as illustrated by FIG. 3C, the blocking material 310-C in the fluid channel 304-C may be heated using a plurality of heat sources 312-1, 312-2, 312-3.

In some examples, the blocking material may act as a heat source, such as a resistive heater. As shown by FIG. 3D, the fluid channel 304-D may include blocking material 310-D which blocks flow of fluid through the fluid channel 304-D and acts as the heat source, with a control line providing an electrical signal to the blocking material 310-D. For example, current may be provided to the blocking material 310-D via a control line and which causes the blocking material 310-D to disintegrate. Example blocking material 310-D includes metal foil and wax (or other material which may be melted) that has silver nanoparticle suspended therein.

FIG. 3E illustrates an example of a fluid blocking region of a fluid channel 304-E that includes breakable material and a piercing structure 314. In such examples, the blocking material 310-E includes the breakable material which may be removed and/or pierced to allow for fluid flow through the fluid channel 304-E by a mechanical force. In some examples, the fluid channel 304-E includes a piercing structure 314 disposed proximal to the blocking material 310-E. A mechanical plunger 313 may apply force on a wall of the fluid channel 310-E which the piercing structure 314 is disposed on and which causes the piercing structure 314 to contact and break the blocking material 310-E. The mechanical plunger 313 may form part of the fluid dispensing device or is separate therefrom. In some examples, the mechanical plunger 313 may be electrically controlled by the fluid dispensing device.

In various examples, the unblocking actuator includes a valve or a vent disposed within each of the plurality of fluid blocking regions which may be coupled to one of the plurality of control lines, as further illustrated by FIGS. 3F-3G. An electrical signal may cause the valve or vent to transition to an open state to allow fluid to flow therethrough and/or allow gas to be removed from respective fluid blocking regions and for fluid to flow therethrough. In some examples, the vent may have a layer of breakable or removable material over the opening of the vent. In some examples, the removable material, such as tape, may be manually removed by the user. In some examples, the layer of breakable material may be pieced manually by the user and/or an electrical signal may be provided to a mechanical component to pierce the breakable material. In other examples, a piercing structure may be located in a fluid dispensing device proximal to where the microfluidic device is disposed or inserted in, and in response to inserting the microfluidic device into the fluid dispensing device, the vents are opened via piercing by the piercing structure.

FIG. 3F illustrates an example of a fluid blocking region of a fluid channel 304-F that includes pressurized gas and a vent 315. In such examples, the blocking material 310-F includes the pressurized gas and the vent 315 may be positioned proximal to the pressured gas, and with each fluid channel of a microfluidic device including include a vent and the pressurized gas. The vent 315 may be in a closed state such that the gas is trapped in the fluid channel 304-F. In some examples, the unblocking actuator and/or unblocking mechanism includes the vent 315 and a layer of breakable material.

In some examples, the vent 315 may include a layer of breakable material over the opening of the vent, which is breakable to allow the pressurized gas to escape the fluid blocking region. The layer of breakable material may be removed or pierced by a user prior to inserting the microfluidic device into the fluid dispensing device, such as by using a mechanical plunger 313 that has a sharp end. In other examples, the fluid dispensing device may include a piercing structure that is actuated to pierce the layer of breakable material and/or that pierces the layer of breakable material in response to inserting the microfluidic device into the fluid dispensing device, as previously described. In other examples, the vent 315 may be electrically actuated. An electrical signal may be provided to the vent 315 by a coupled control line, which causes the vent 315 to transition from the closed state to an open state, such that pressurized gas escapes the fluid blocking region and fluid may flow through the fluid blocking region and to the dehydrated reagent downstream.

FIG. 3G illustrates an example of a fluid blocking region of a fluid channel 304-G that includes a valve. In some examples, the blocking material 310-G may include or form the valve positioned across fluid channel 304-G in the fluid blocking region. The valve may be opened and/or closed on command, such as in response to electrical signal applied thereto. In the closed state, as shown at 323, the valve blocks the flow of fluid. In the open state, as shown at 325, the valve allows fluid to flow through.

In such examples, the unblocking actuator and/or mechanism includes a pneumatic source 316 which is coupled to the plurality of fluid channels and/or coupled to the fluid channel 304-G of the plurality, and in which the microfluidic device includes a plurality of pneumatic sources. An electrical signal may be provided to the pneumatic source 316 that selectively provides a pneumatic signal 317 to select ones of the plurality of fluid blocking regions in response. In some examples, the pneumatic signal 317 is provided along ones of the fluid channels toward the respective blocking material(s). The pneumatic signal 317 may cause the blocking material to change states and unblock the respective fluid channel.

In some examples and as shown by FIG. 3G, the valve includes a membrane formed of a flexible material. In a first state, as shown at 323, the valve is deflected (downward in the orientation of FIG. 3G) to close the fluid channel 304-G. The pneumatic source 316 applies a pneumatic signal 317, such as a pressurized air or other gas, to the fluid channel 304-G, which causes the valve to transition from the first state to a second state, as shown at 325, in which the valve is pushed out the way by the pneumatic signal 317, such as upward. An electrical signal may be provided to the pneumatic source 316 by a coupled fluid dispensing device, and in response, the pneumatic source 316 applies the pneumatic signal 317 to the fluid channel 304-G.

In some examples, the valve may be otherwise electrically control via a control line. For example, the microfluidic device may include a plurality of valves disposed within the plurality of fluid blocking regions. The valve may be in the first state, in which the fluid channel is closed or blocked by the valve, via a coupled magnet or latch. An electrical signal provided to the valve may cause the valve to unlatch and/or another magnet, which is stronger than the coupled magnet may be activated to cause the valve to transition to a second state in which the fluid channel is open and fluid may flow through. The other magnet may include an electromagnet which is activated by the electrical signal and/or a magnet of the fluid dispensing device which is brought in proximity to the fluid channel by the moveable component in response to the electrical signal.

In some examples, and as previously illustrated by FIG. 1B and FIGS. 3A-3D, an irreversible valve may be formed using material which melts and/or transitions between a solid state and a fluid or gas state. The blocking material may be melted or chemically reacted to remove the material from the fluid channel. Such valves may include no moving parts other than the fluid in the fluid channel. In some examples, no external components are used to actuate the valves, other than a source of an electric signal.

In various examples, the blocking material may include a magnetic material or ferrofluid. A plurality of magnets may be coupled to a mechanical component that selectively moves a respective magnet causing the magnetic material to move within the fluid channel and unblocking the fluid channel due to movement of a magnetic field. In some examples, the mechanical component may cause the movement of the magnet in response to an electrical signal provided thereto. In other examples, the plurality of magnets include electromagnets which are activated to provide a magnetic field in response to an electrical signal provided thereto.

FIG. 3H illustrates an example of a fluid blocking region of a fluid channel 304-H that includes magnetic material, such as a permanent magnet. In such examples, the blocking material 310-H includes the magnetic material, and the unblocking actuator and/or unblocking mechanism includes a magnet 318 that provides a magnetic field to the magnetic material to move the magnetic material. In some examples, each fluid channel of a microfluidic device includes magnetic material. In some examples, the fluid channel 304-H may further include a side chamber 319 coupled to the fluid blocking region, and in which the magnetic material may be moved to. In some examples, the magnet 318 includes an electromagnet which is activated and outputs the magnetic field in response to an electrical signal. The electromagnet may be disposed within or proximal to the side chamber 319. In some examples, the magnet 318 is a component of the fluid dispensing device, and is brought in proximity to the fluid blocking region to move the magnetic material. The magnetic material may be disposed within the fluid blocking region in a first state (e.g., closed state), as shown at 327, that blocks fluid flow, and in response to the magnetic field applied by the magnet 318, the magnetic material transition to a second state (e.g., open state) in which fluid may flow through the fluid channel, as shown at 329. In some examples, the second state includes the blocking material 310-H in the side chamber 319 and, in other examples, includes a different orientation such that the blocking material 310-H is partially blocking the fluid flow.

FIG. 3I illustrates an example of a fluid blocking region of a fluid channel 304-I that includes ferrofluid. In such examples, the blocking material includes 310-I the ferrofluid, and the unblocking actuator and/or unblocking mechanism includes an electromagnet 320 that provides a magnetic field to the ferrofluid to move the ferrofluid. In some examples, each fluid channel of a microfluidic device includes ferrofluid. As described above, the electromagnet 320 may be activated and outputs the magnetic field in response to an electrical signal applied thereto. In some examples, a side chamber 319 is coupled to the fluid blocking region. The ferrofluid may be disposed within the fluid blocking region in a first state (e.g., closed state) that blocks fluid flow, as shown at 331, and in response to the magnetic field applied by the electromagnet 320, the ferrofluid transitions to a second state (e.g., open state) in which fluid may flow through the fluid channel, such as moving into the side chamber 319 as shown at 333.

FIG. 3J illustrates an example of a fluid blocking region of a fluid channel 304-J that includes a capillary chamber and fluid actuator 321. In such examples, the blocking material 310-J includes the capillary chamber (which forms part of the fluid channel 304-J), and the unblocking actuator and/or unblocking mechanism includes the fluid actuator 321 and a control line that provides an electric signal to the fluid actuator 321 to cause actuation of fluid.

The capillary chamber has a different dimension (e.g., width, height, diameter) than the remaining portions of the fluid channel 304-J. Fluid may be passively flowed through the fluid channels due to capillary forces. The change in dimensions between the capillary chamber (e.g., blocking material 310-J) and the remaining portions of fluid channel 304-J may cause disruption in the fluid flow, such that the flow of fluid from the capillary forces is prevented at an inlet due to the capillary chamber. A fluid actuator 321 may be located proximal to the inlet of the capillary chamber and disposed within each of the fluid channels, such as a resistor or piezoelectric membrane as previously described. The electrical signal may be provided to the respective fluid actuator 321 by a coupled control line, which causes the fluid actuator 321 to actuate and to draw fluid into the capillary chamber and allows for the flow of fluid through the fluid blocking region and to the dehydrated reagent downstream.

FIG. 3K illustrates an example of a fluid blocking region of a fluid channel 304-K that includes hollow capsule formed of glass. In some examples, the fluid channel 304-K may include flexible walls and the blocking material 310-K may include the hollow capsule. The unblocking actuator and/or unblocking mechanism may include a pneumatic source or other source of pressure, such as the illustrated mechanical plunger 313, which provides pressure on the flexible walls of the fluid channel 304-K proximal to the hollow capsule. The pressure causes the capsule to break and the fluid channel to be unblocked.

Example blocking material and unblocking actuators and/or mechanisms are not limited to that illustrated and may include a variety of variations and/or combinations of that illustrated and described herein.

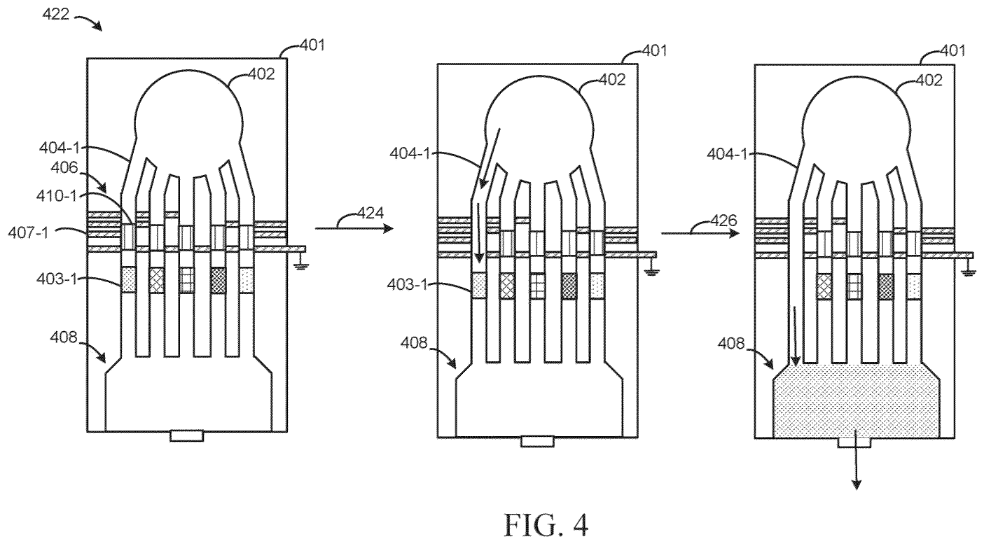

FIG. 4 illustrates an example operation of a microfluidic device with dehydrated reagents, in accordance with the present disclosure. The microfluidic device 401 may include an implementation of and/or include similar features and components as the microfluidic device 101 of FIG. 1B, and is numbered accordingly. For instance, the microfluidic device 401 includes a plurality of fluid channels (as illustrated by the labeled fluid channel 404-1) having a plurality of fluid blocking regions with blocking material (as illustrated by the labeled blocking material 410-1) and a plurality of dehydrated reagents (as illustrated by the labeled dehydrated reagent 403-1), the unblocking actuator 406 including the plurality of control lines (as illustrated by the labeled control line 407-1), and the fluid ejection device 408.

For the operation 422 of the microfluidic device 401, a user may select a first dehydrated reagent 403-1 of the plurality of dehydrated reagents to be ejected from the microfluidic device 401. The selection may be based on a prior test performed, such as based on an analysis of how the sample fluid reacted to prior deposited reagents which may have been ejected by the microfluidic device 401 or another one. However, examples are not so limited. The user may provide the input to a fluid dispensing device which is coupled to the microfluidic device 401 and which controls the operation of the microfluidic device 401.

In response to the selection, at 424, an electrical signal is applied and transmitted across the first control line 407-1 coupled to the first fluidic blocking region proximal to the blocking material 410-1. In some examples, the blocking material 410-1 includes material which transitions states in response to heat applied thereto. For example, the control line 407-1 includes a resistor trace line which carries the electrical signal and causes heat to be applied to the blocking material 410-1. The blocking material 410-1 may melt to a liquid state or transitions to a gas state, which unblocks the first fluid channel 404-1 and allows fluid communication between the first fluid chamber 402 and the first dehydrated reagent 403-1. In some examples, a force is applied to the reservoir coupled to the first fluid chamber 402 to provide fluid to the first fluid chamber 402. The fluid flows along the first fluid channel 404-1 to the first dehydrated reagent 403-1 and reconstitutes the first dehydrated reagent 403-1. At 426, the fluid with the reconstituted first dehydrated reagent 403-1 flows to the first ejection device 408 and is ejected to a substrate through the nozzle, as illustrated by the arrow.

As an example, such as where the reservoir includes a blister pack, a plunger may press on the blister with the fluid therein, driving the fluid through the first fluid channel 404-1 and to the first dehydrated reagent 403-1, which is reconstituted in the fluid. Although examples are not so limited, and the blister pack may be actuated in response to the removal of the blocking material 410-1, such as with a blister pack that is under pressure. In other examples and/or in addition, the fluid flow may be actuated via a fluid actuator disposed proximal to the first fluid channel 404-1 and/or the first fluid chamber 402.

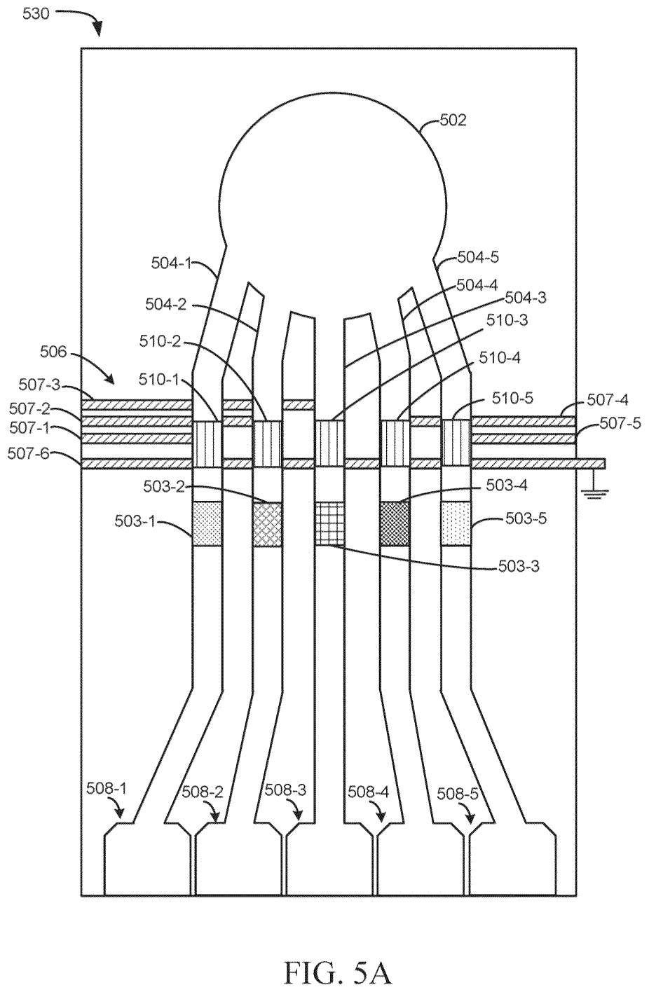

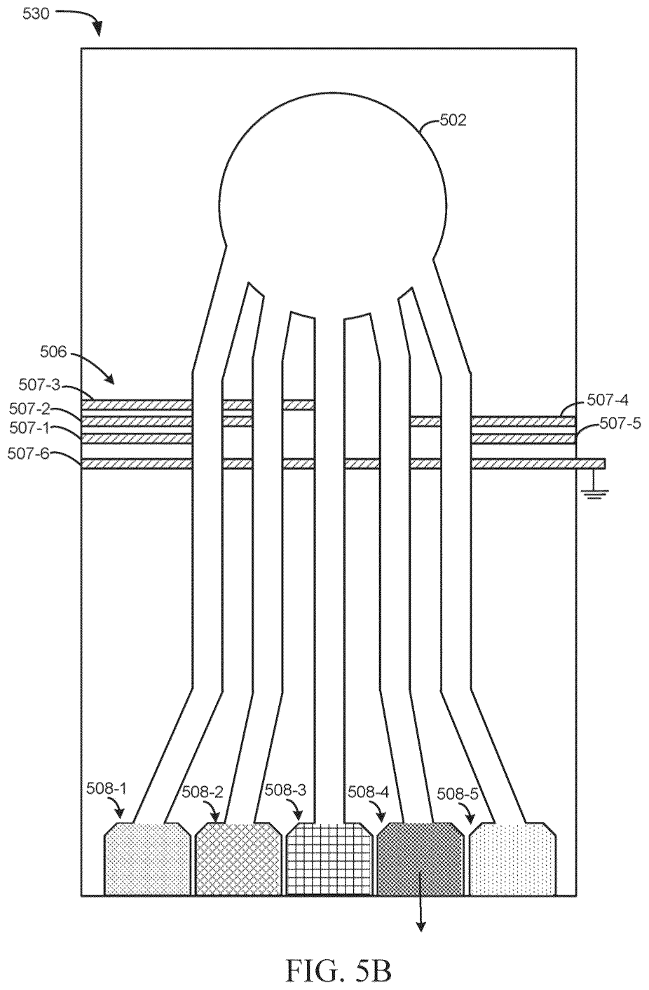

Microfluidic devices may include variations from that illustrated above, such as additional or fewer fluid channels, dehydrated reagents, and blocking material, chambers, fluid actuators, shapes and dimensions. In some examples, the fluid ejection device 408 may include a plurality of fluid ejection devices including a plurality of second fluid chambers, wherein each of the plurality of second fluid chambers are fluidically coupled to a respective fluid channel of the plurality of fluid channels, and each of the plurality of fluid ejection devices further include a fluid actuator and nozzle to eject fluid therefrom, as further illustrated by FIGS. 5A-5B.

FIGS. 5A-5B illustrate another example microfluidic device with dehydrated reagents and a plurality of dedicated fluid ejection devices, in accordance with the present disclosure. The microfluidic device 530 of FIGS. 5A-5B may include an implementation of and/or include similar features and components as the microfluidic devices of FIG. 1B, with variations including a plurality of fluid ejection devices coupled to a respective one of the plurality of fluid channels and is numbered accordingly.

The microfluidic device 530 of FIGS. 5A-5B include a plurality of fluid channels 504-1, 504-2, 504-3, 504-4, 504-5 (herein generally referred to as “the plurality of fluid channels 504” for ease of reference) having a plurality of fluid blocking regions with a plurality of blocking material 510-1, 510-2, 510-3, 510-4, 510-5 (herein generally referred to as “the plurality of blocking material 510” for ease of reference) and a plurality of dehydrated reagents 503-1, 503-2, 503-3, 503-4, 503-5 (herein generally referred to as “the plurality of dehydrated reagents 503” for ease of reference), an unblocking actuator 506 that includes a plurality of control lines 507-1, 507-2, 507-3, 507-3, 507-4, 507-5, 507-6 (herein generally referred to as “the plurality of control lines 507” for ease of reference) and a plurality of fluid ejection devices 508-1, 508-2, 508-3, 508-4, 508-5 (herein generally referred to as “the plurality of fluid ejection devices 508” for ease of reference). In some examples, the microfluidic device 530 further includes a first fluid chamber 502 fluidically coupled to the plurality of fluid channels 504 for receiving fluid, such as from a coupled reservoir. The microfluidic device 530 may include the various variations previously described, and in various combinations, including the different types of blocking material, different numbers of blocking regions (e.g., upstream and downstream from the dehydrated reagents as illustrated by FIG. 3A), among others.

As shown by FIG. 5A, each fluid channel of the plurality of fluid channels 504 is coupled to a respective fluid ejection device of the plurality of fluid ejection devices 508. In some examples, each of the plurality of fluid ejection device 508 includes a second fluid chamber, which provides an entrance to the die of the fluid ejections devices 508.

FIG. 5B illustrates an example operation of the microfluidic device 530 of FIG. 5A. In some examples, each of the plurality of blocking material (510 referring to FIG. 5A) may be removed to allow fluid to flow through each of the plurality of fluid channels 504 via the unblocking actuator 506, and to reconstitute each of the plurality of dehydrated reagents 503 within the fluid and to flow each of the reconstituted rehydrated reagents to a respective one of the plurality of fluid ejection devices 508 (via a fluid control actuator). Reagents may subsequently be selected for ejection, and the fluid ejection device associated with the selected reagent, such as fluid ejection device 508-4, is actuated to eject the reconstituted dehydrated reagent from the microfluidic device 530. In some examples, the microfluidic device 530 may be used to eject different concentrations of each reagent and/or different mixtures of the reagents.

Examples are not so limited and in some examples, the fluid channels 504 may be unblock sequentially, rather than concurrently and/or all together.

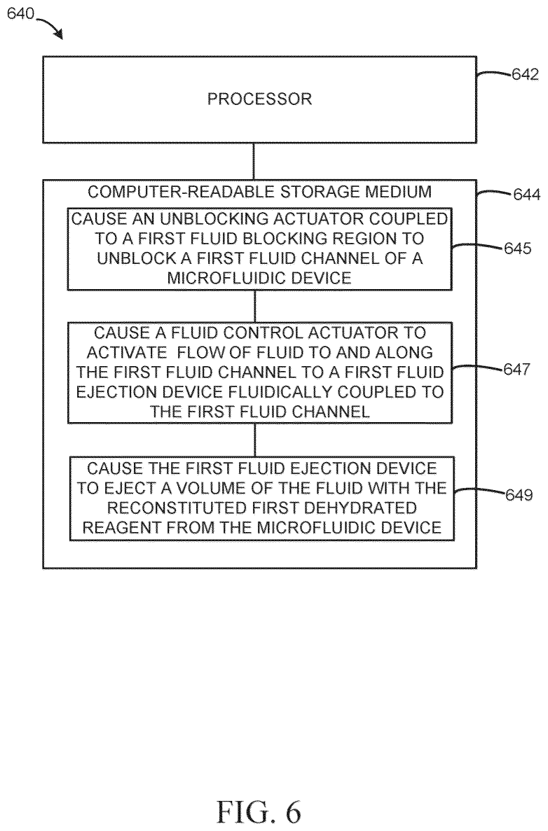

FIG. 6 illustrates an example device including non-transitory computer-readable storage medium, in accordance with examples of the present disclosure. The device 640 includes a processor 642 and memory. The memory may include a computer-readable storage medium 644 storing a set of instructions 645, 647, 649. In some examples, the device 640 is the fluid dispensing device in which a microfluidic device, as described herein, is disposed within.

The computer-readable storage medium 644 may include Read-Only Memory (ROM), Random-Access Memory (RAM), Electrically Erasable Programmable Read-Only Memory (EEPROM), flash memory, a solid state drive, Electrically Programmable Read Only Memory aka write once memory (EPROM), physical fuses and e-fuses, and/or discrete data register sets. In some examples, computer-readable storage medium 644 may be a non-transitory storage medium, where the term “non-transitory” does not encompass transitory propagating signals.

At 645, the processor 642 may cause an unblocking actuator coupled to a first fluid blocking region of a plurality of fluid blocking regions to unblock a first fluid channel of a plurality of fluid channels of a microfluidic device. The microfluidic device may include substantially the same features and components as previously described at least by FIGS. 1A-5B, the features and components of which are not repeated. As previously described and in some examples, the processor 642 may apply an electrical signal to a control line which is coupled to the first fluid blocking region and/or to another component, such as a valve, vent, a pneumatic source, a magnet, or other mechanical component, to cause unblockage of a fluid channel. In some examples, the unblocking actuator includes the control line. For example, the electrical signal may be transmitted across the control line and cause heat to be transmitted to a first blocking material of the first fluid blocking region or activates the first blocking material (e.g., a valve) or other component coupled to the first blocking material, such as a vent, a fluid actuator, or another heat source. In some examples, the electrical signal is transmitted over the control line (e.g., a resistor trace line) that is disposed proximal to the first fluid blocking region and which causes application of heat to blocking material disposed within the first fluid blocking region.

At 647, the processor 642 may cause a fluid control actuator to activate flow of fluid to and along the first fluid channel to a first fluid ejection device fluidically coupled to the first fluid channel. In some examples, the processor 642 may send or transmit an electrical signal to the fluid control actuator to cause activation of the fluid flow. Fluid flow may be activated prior to, concurrently with, or after the unblocking of the first fluid channel. In some examples, fluid flow may be activated from a reservoir to a first fluid chamber of the microfluidic device, to and along the first fluid channel, and to the first fluid ejection device. As previously described, the flow of fluid reconstitutes a first dehydrated reagent of the plurality of dehydrated reagents within the fluid.

In some examples, the fluid control actuator may form part of the microfluidic device and/or the fluid dispensing device. For example, the fluid control actuator may include a plunger or other force source, a coupled reservoir (e.g., blister pack), the electrical signal itself, and/or a fluid actuator disposed within a fluid chamber of the microfluidic device (e.g., the first fluid chamber) and/or a plurality of fluid actuators disposed within the fluid channels.

In some examples, the fluid control actuator includes a plunger of the fluid dispensing device which is disposed proximal to the microfluidic device. The processor 642 transmits an electrical signal to the plunger to cause the plunger to move and apply a force to the reservoir. For example, the processor 642 may activate the plunger disposed proximal to a reservoir to press on the reservoir, and in response, break a layer of breakable material of the reservoir and provide fluidic coupling between the reservoir and a first fluid chamber fluidically coupled to the plurality of fluid channels. In some examples, the activation of the flow of fluid causes the fluid to flow from the first fluid chamber to each of the plurality of fluid blocking regions, and the remaining plurality of fluid blocking regions block the flow of fluid.

However, examples are not so limited and may include different variations as described above. In some examples, the reservoir is pressurized and unblocking of the first fluid channel may cause a change in pressure, and breaking of the layer of breakable material. In such examples, the unblocking actuator is the fluid control actuator. In other examples and/or in addition, the processor 642 may transmit another electrical signal to a vent or a valve disposed within the first fluid blocking region and/or proximal to the first blocking material, and which instructs the vent or valve to transition states (e.g., from a closed position to an open position) to remove the first blocking material from the first fluid channel and to unblock the first fluid channel. In other examples, the processor 642 may transmit another electrical signal to a fluid actuator disposed within the first fluid blocking region and/or proximal to the first blocking material, which causes the fluid actuator to actuate and the fluid flow.

At 649, the processor 642 may cause the first fluid ejection device to eject a volume of the fluid with the reconstituted first dehydrated reagent from the microfluidic device. The fluid may be ejected via an ejection chamber with a fluid actuator and a coupled nozzle. As previously described, the fluid actuator may include a resistor, and the processor 642 may actuate the resistor of the first fluid ejection device to cause the ejection of the volume of the fluid by transmitting or applying an electrical signal to the resistor.

In some examples, the volume of fluid is ejected from the microfluidic device to a region of a substrate and the processor 642 causes a fluid ejection device of a second microfluidic device to eject a volume of sample fluid to the region of the substrate. For example, the first microfluidic device and the second microfluidic device may be disposed within the fluid dispensing device for ejecting the different fluids. However, examples are not so limited and the sample fluid may be pre-deposited on the substrate, such as by plating bacterium on an agar plate.

In some examples, the processor 642 may cause the unblocking actuator coupled to a second fluid blocking region of the plurality of fluid blocking regions to unblock a second fluid channel of the plurality of fluid channels. For example, the processor 642 may apply a second electrical signal to a second control line coupled to the second fluid blocking region. The activation of the flow of fluid may cause the fluid to flow along the second fluid channel and to the first fluid ejection device fluidically coupled to the second fluid channel and to reconstitute the second dehydrated reagent of the plurality of dehydrated reagents within the fluid, and the volume of the fluid ejected includes the reconstituted first dehydrated reagent and the reconstituted second dehydrated reagent. For example, a microfluidic device may have a shared fluid ejection device, such as microfluidic device 101 of FIG. 1B, which is used to mix at least two reagents in a second fluid chamber that forms part of the package of the die of the fluid ejection device.

In some examples, the processor 642 may cause the unblocking actuator coupled to a second fluid blocking region of the plurality of fluid blocking regions to unblock a second fluid channel of the plurality of fluid channels, such as by applying a second electrical signal to a second control line. In some examples and as described above, the unblocking actuator includes a plurality of control lines and the processor 642 applies a first electrical signal to a first control line of the plurality of control lines and a second electrical signal to a second control line of the plurality of control lines. The first electrical signal and the second electrical signal may cause application of heat to the first blocking region and the second blocking region. In some examples, the activation of the flow of fluid causes the fluid to flow along the second fluid channel and to a second fluid ejection device fluidically coupled to the second fluid channel, and to reconstitute the second dehydrated reagent of the plurality of reconstituted reagents within the fluid, and the processor 642 further causes the second fluid ejection device to eject a volume of the fluid with the reconstituted second dehydrated reagent from the microfluidic device. In some examples, the processor 642 may provide an electrical signal to each of the plurality of control lines, such as for a microfluidic device having a dedicated fluid ejection device for each fluid channel as illustrated by microfluidic device 530 of FIG. 5A.

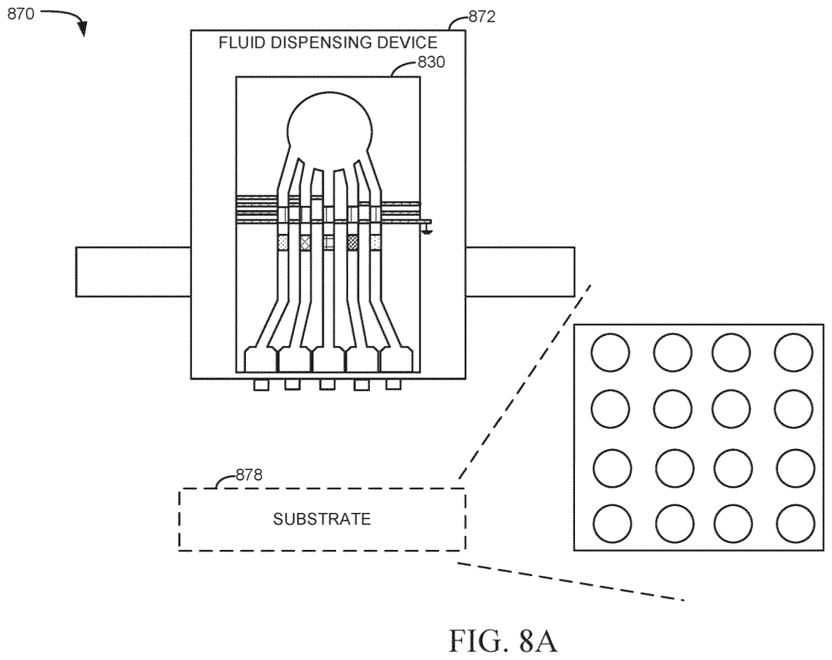

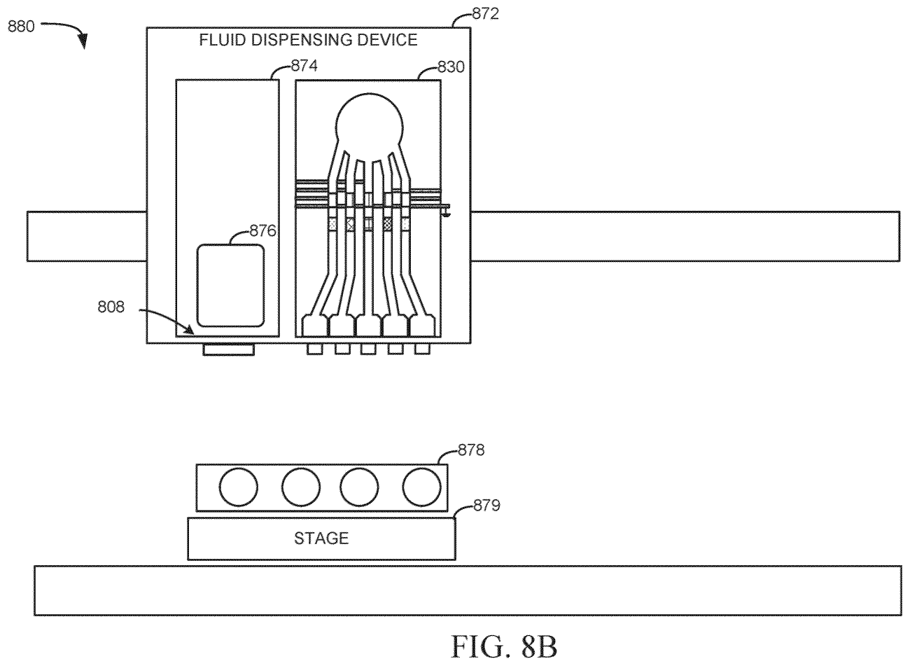

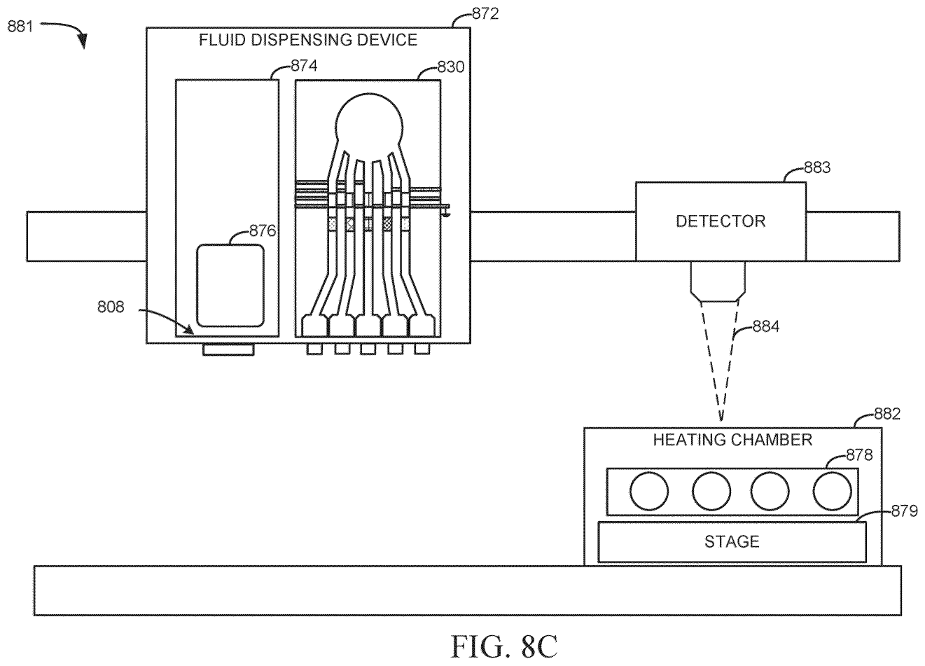

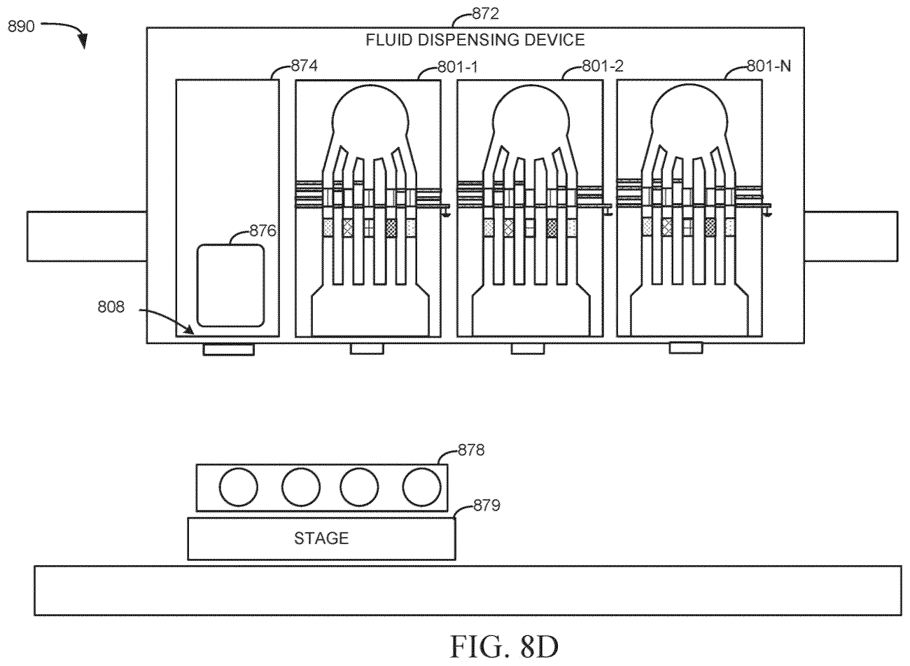

In various examples, the device 640 may be used to control the operation of the plurality of microfluidic devices, which as disposed within the device 640, as further illustrated herein by FIGS. 8A-8D.

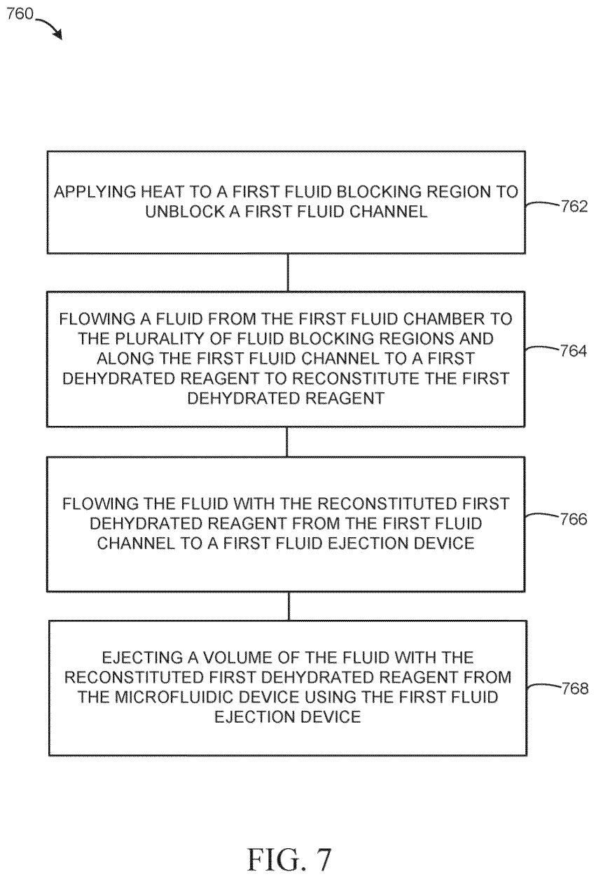

FIG. 7 illustrates an example method for ejecting reconstituted dehydrated reagent from a microfluidic device, in accordance with examples of the present disclosure. The method may be implemented by or using any of the microfluidic devices as illustrated by FIGS. 1A-1B, FIG. 3, FIGS. 5A-5B, a device 640 as illustrated by FIG. 6, and/or by the systems of FIGS. 8A-8D.

At 762, the method 760 includes applying heat to a first fluid blocking region of a plurality of fluid blocking regions to unblock a first fluid channel of a plurality of fluid channels of a microfluidic device. As previously described, the plurality of fluid channels include a plurality of dehydrated reagents disposed within the plurality of fluid channels, and the plurality of fluid blocking regions include blocking material disposed within the plurality of fluid channels between a first fluid chamber of the microfluidic device and the plurality of dehydrated reagents to block flow of the fluid along the plurality of fluid channel. At 764, the method 760 includes flowing a fluid from the first fluid chamber to the plurality of fluid blocking regions and along the first fluid channel to a first dehydrated reagent of the plurality of dehydrated reagents disposed within the first fluid channel to reconstitute the first dehydrated reagent within the fluid. At 766, the method 760 includes flowing the fluid with the reconstituted first dehydrated reagent from the first fluid channel to a first fluid ejection device of the microfluidic device.

At 768, the method 760 includes ejecting a volume of the fluid with the reconstituted first dehydrated reagent from the microfluidic device using the first fluid ejection device. In some examples, the volume of fluid is ejected from the microfluidic device to a region of a substrate, and the method 760 further includes ejecting a volume of sample fluid to the region of the substrate using a fluid ejection device of a second microfluidic device, wherein the microfluidic device and the second microfluidic device are coupled to and disposed within a fluid dispensing device. However, examples are not so limited and the sample fluid may be pre-deposited on the substrate in some examples.

In some examples, the method 760 includes applying heat to a second fluid blocking region of a plurality of fluid blocking regions to unblock a second fluid channel of the plurality of fluid channels, flowing the fluid from the first fluid chamber and along the second fluid channel to a second dehydrated reagent of the plurality of dehydrated reagents disposed within the second fluid channel to reconstitute the second dehydrated reagent within the fluid, and flowing the fluid with the reconstituted second dehydrated reagent from the second fluid channel to one of the first fluid ejection device and a second fluid ejection device of the microfluidic device. The method 760 further includes ejecting a volume of the fluid with the reconstituted second dehydrated reagent from the microfluidic device using the one of the first fluid ejection device and the second fluid ejection device. The volume with the reconstituted first dehydrated reagent and the volume with the reconstituted second dehydrated reagent may be ejected concurrently or sequentially using the same fluid ejection device or different fluid ejection devices. And, the application of heat to the first fluid blocking region and the second fluid blocking region may occur concurrently or sequentially.

In some examples, the fluid flow may be caused by breaking a layer of breakable material of a reservoir containing the fluid. For example, the method 760 may include breaking the layer of breakable material of the reservoir containing the fluid to fluidically couple the reservoir to the first fluid chamber and, in response, flowing the fluid from the first fluid chamber to the plurality of fluid blocking regions. As previously described, the reservoir may include a blister pack coupled to the first fluid chamber. In some examples, the blister pack is under pressure, and applying the force to the first fluid blocking region and unblocking the first fluid channel causes the break in the layer of breakable material. In other examples, a plunger may be activated to break the layer of breakable material, as previously described.

In some examples, the microfluidic device is disposed within a fluid dispensing device with a plurality of microfluidic devices, and each of the plurality of microfluidic devices include respective fluid channels fluidically coupled to first fluid chamber and to fluid ejection devices. The method 760 may further include selectively applying heat to respective fluid blocking regions of the plurality of microfluidic device, flowing respective fluids from the first fluid chambers to the fluid ejection devices, and ejecting fluids from the plurality of microfluidic devices which include different combinations of the plurality of reagents as reconstituted in the ejected fluids.

Examples are not so limited and methods may include other ways of removing blocking material and/or other types of blocking material, such as those illustrated by FIGS. 3A-3K.

FIGS. 8A-8D illustrate different example systems including a fluid dispensing device and a first microfluidic device with dehydrated reagents, in accordance with the present disclosure.

A fluid dispensing device may be an ink-jet based device that may dispense picoliters or nanoliters of fluid into specific locations on a substrate. In various examples, a fluid dispensing device may use a microfluidic device (e.g., a cartridge) as described herein for dispensing fluid. The microfluidic device may operate similar to a printhead. The fluid dispensing device may include a substrate transport assembly to move the substrate and circuitry, such as the processor and memory as previously described by FIG. 6, the components of which are not repeated or illustrated. The fluid dispensing device may include additionally non-illustrated components, such as a mounting assembly and a power supply that provides power to the various electrical components of the fluid dispensing device and the microfluidic device mounted therein.

As shown by FIG. 8A, an example system 870 includes a fluid dispensing device 872 and a microfluidic device 830. The microfluidic device 830 may include an implementation of and/or include similar features and components as any of the microfluidic devices 100, 101, 530 of FIGS. 1A-1B and 5A, the common features and components being illustrated but not repeated. As previously described, in some examples, the microfluidic device 830 includes a plurality fluid ejection devices that drop fluid through nozzles toward a substrate 878. Each fluid ejection device may dedicated to a respective fluid channel of the microfluidic device 830.

However examples are not so limited, and the microfluidic device 830 of the system 870 may include any of the microfluidic devices described herein.

The fluid dispensing device 872 is coupled to the microfluidic device 830. The fluid dispensing device 872 includes a processor to cause a first fluid blocking region of the plurality of fluid blocking regions to unblock a first fluid channel of the plurality of fluid channels of the microfluidic device 830, and cause a fluid control actuator to activate flow of fluid to and along the first fluid channel and to the fluid ejection device fluidically coupled to the first fluid channel, wherein the flow of fluid reconstitutes a first dehydrated reagent of the plurality of dehydrated reagents within the fluid. The processor further causes the fluid ejection device to eject a volume of the fluid with the reconstituted first dehydrated reagent from the microfluidic device 830.

In the particular example, the fluid dispensing device 872 may unblock each of the plurality of fluid channels of the microfluidic device 830 and eject different amounts, ratios, and/or combinations of a plurality of reconstituted rehydrated reagents to the substrate 878. In some examples, the system 870 includes the substrate 878. The substrate 878 may include different regions, such as wells of a well plate, with each region getting a different amount of a reagent and/or different mixture of the reagents. In some examples, each region of the substrate 878 may include the sample fluid and, in other examples, the fluid dispensing device 872 may dispense the sample fluid to each region of the substrate 878. These dispense locations may be specific target regions on the substrate surface, such as cavities, microwells, channels, indentation into the substrate, or other regions of the substrate. As used herein, a microwell includes and/or refers to a column capable of storing a volume of fluid between a nanoliter and several milliliters of fluid.

As an example, the dehydrated reagents of the microfluidic device 830 may include four different antibiotics and a metabolic indicator. The substrate 878 may include a 16-well microplate. Each of the wells of the substrate 878 may contain an equal amount of bacteria inoculum and metabolic indicator. The metabolic indicator may be ejected from the microfluidic device 830 and the bacteria inoculum may be ejected from a second microfluidic device via control by the fluid dispensing device 872, such as further illustrated FIG. 8B. In some examples, the bacteria may be pre-plated on the substrate 878, such as with an agar plate. Each column of wells of the 16-well microplate may be associated with a particular antibiotic of the four. For example, the fluid dispensing device 872 may control ejection of a first antibiotic into wells of the first column of the microplate, ejection of a second antibiotic into wells of the second column of the microplate, ejection of a third antibiotic into wells of the third column of the microplate, and ejection of a fourth antibiotic into wells of the fourth column of the microplate. Each row of a column may have a different amount (e.g., concentration) of the respective antibiotic, and in some examples, a row may include a control group that has no antibiotic. Such reagents may be used to test antibiotic susceptibility.

In some examples, regions of the substrate 878 may contain mixtures of at least two antibiotics. For example, the antibiotics may be ejected to form gradients of mixtures of the antibiotics at different ratios and/or concentrations. The gradients may be used to measure both antibiotic susceptibility response and a response curve. Examples are not limited to antibiotics, and may include other types of reagents and sample fluid which may be tested to identify cells within the sample fluid that survive or are killed, among other types of tests.

FIG. 8B illustrates another example system 880. The system 880 of FIG. 8B may include an implementation of and/or include similar features and components of the system 870 of FIG. 8A, with the addition of a second microfluidic device and a substrate transport assembly. For instance, the system 880 includes a fluid dispensing device 872 and a first microfluidic device 830. The common features and components are not repeated.

The system 880 further includes a second microfluidic device 874. The second microfluidic device 874, similar to the first microfluidic device 830, may operate as or similar to a printhead. The second microfluidic device 874 includes a fluid chamber 876 and fluid ejection device 808 coupled to the fluid chamber 876. The fluid chamber 876 contains a sample fluid. The fluid dispensing device 872 may cause flow of the sample fluid from the fluid chamber 876 to the fluid ejection device 808 and cause the fluid ejection device 808 (e.g., actuate a resistor in an ejection chamber coupled to a nozzle) to eject a volume of the sample fluid from the fluid ejection device 808 to a region of the substrate 878, such as to a plurality of regions of the substrate 878.

The system 880 further includes a substrate transport assembly which includes a stage 879 coupled to one of the substrate 878 and the fluid dispensing device 872 to move a position of the substrate 878 with respect to the fluid dispensing device 872.

FIG. 8C illustrates a further example system 881. The system 881 of FIG. 8C may include an implementation of and/or include similar features and components of the system 880 of FIG. 8B, with the addition of a heating chamber 882 and a detector 883. For instance, the system 881 includes a fluid dispensing device 872, a first microfluidic device 830, a second microfluidic device 874, a substrate 878, and a substrate transport assembly including a stage 879. The common features and components are not repeated.

The system 881 further includes a heating chamber 882 or other heat source and a detector 883. The stage 879 may move the substrate 878 from a position proximal to the fluid dispensing device 872 to a position proximal to the heating chamber 882. For example, the stage 879 may move the substrate 878 such that the substrate 878 is contained within the heating chamber 882. The heating chamber 882 may heat the plurality of regions of the substrate 878 to drive a biochemical reaction (e.g., incubation) between the ejected reagents and the sample fluid.

The detector 883 may be disposed proximal to the heating chamber 882 to detect the reactions occurring in the plurality of regions of the substrate 878, and optionally, while the reactions are occurring. The heating chamber 882 may include a transparent lid such that excitation light 884 may be provided by the detector 883 to the plurality of regions of the substrate 878 and light emitted from the plurality of regions of the substrate 878 in response to the excitation light 884 is provided back to the detector 883. In some examples, the detector 883 may be used to provide a measure of fluorescence. For example, as the reagents react with the sample fluid, the metabolic indicator may be converted to a fluorescent compound, which is detected via the detector 883 as a fluorescent signal.

FIG. 8D illustrates another example system 890. The system 890 of FIG. 8D may include an implementation of and/or include similar features and components of the system 880 of FIG. 8B and/or system 881 of FIG. 8C, with additional first microfluidic devices. For instance, the system 890 includes a fluid dispensing device 872, a first microfluidic device 801-1, a second microfluidic device 874, a substrate 878, and a substrate transport assembly including a stage 879. The common features and components are not repeated.

The system 890 includes a plurality of first microfluidic devices 801-1, 801-2, 801-N which are disposed within the fluid dispensing device 872. Each of the plurality of first microfluidic devices 801-1, 801-2, 801-N may include an implementation of and/or include similar features and components as any of the microfluidic devices 100, 101, 530 of FIGS. 1A-1B and 5A, the common features and components being illustrated but not repeated.

In some examples, each of the plurality of first microfluidic devices 801-1, 801-2, 801-N includes a different set of dehydrated reagents. The different sets of dehydrated reagents may include different reagents and/or different concentrations of reagents. Using the particular example, three first microfluidic devices 801-1, 801-2, 801-N are loaded onto the fluid dispensing device 872. Each of the first microfluidic devices 801-1, 801-2, 801-N includes five different antibiotics in the five fluid channels. Within each microfluidic device 801-1, 801-2, 801-N, mixtures of different combinations of the five antibiotics converge at a second fluid chamber that forms part of or is on the package of the respective fluid ejection device. The mixtures are ejected out into regions of the substrate 878 which contains a sample fluid, such as bacterium inoculum. With five reagents in the fluid channels for three different microfluidic devices 801-1, 801-2, 801-N, if one reagent is dispensed per channel, there are 5\hat{ }3 different mixture combinations, for a total of 125 possible combinations. By increasing to five microfluidic devices, there is 3125 possible combinations. With five fluid channels and five reagents, and with more than one reagent being dispensed per channel, there are further combinations possible, such as:

$${( ( \frac{5}{1} ) + ( \frac{5}{2} ) + ( \frac{5}{3} ) + ( \frac{5}{4} ) + ( \frac{5}{5} ) )}^{5} = 28629151 .$$

The fluid dispensing device 872 may be used to dispense different sets of tests using additional microfluidic devices. As an example, results from a prior test may be used to select reagents to use in subsequent test. As an example, for antibiotic susceptibility test, bacterial resistance profiles may be determined using relatively small amounts of bacteria.

Circuitry as used herein, such as processor 642 include a processor, computer readable instructions, and other electronics for communicating with and controlling the heater(s), and other components of the apparatus, such as a fluid actuator(s) and/or resistor(s), and other components. The circuitry may receive data from a host system, such as a fluid dispensing device, and includes memory for temporarily storing data. The data may be sent to the apparatus along an electronic, infrared, optical, or other information transfer path. A processor may be a central processing unit (CPU), a semiconductor-based microprocessor, a graphics processing unit (GPU), a microcontroller, special purpose logic hardware controlled by microcode or other hardware devices suitable for retrieval and/or execution of instructions stored in a memory, or combinations thereof. In addition to or alternatively to retrieving and executing instructions, the processor may include at least one integrated circuit (IC), other control logic, other electronic circuits, or combinations thereof that include a number of electronic components for performing the function. In some examples, the circuitry includes non-transitory computer-readable storage medium that is encoded with a series of executable instructions that may be executed by the processor. Non-transitory computer-readable storage medium may be an electronic, magnetic, optical, or other physical storage device that contains or stores executable instructions. Thus, non-transitory computer-readable storage medium may be, for example, RAM, an EEPROM, a storage device, an optical disc, etc. In some examples, the computer-readable storage medium may be a non-transitory storage medium, where the term ‘non-transitory’ does not encompass transitory propagating signals.

A sample and/or sample fluid, as used herein, refers to and/or includes any material, collected from a subject, such as biologic material. Example samples include whole blood, blood plasma, and other body fluids, as well as tissue cell cultures obtained from humans, plants, or animals. Such samples may contain viral or cellular material, including all prokaryotic or eukaryotic cells, viruses, bacteriophages, mycoplasmas, protoplasts, and organelles. Biological material may comprise all types of mammalian and non-mammalian animal cells, plant cells, algae including blue-green algae, fungi, bacteria, protozoa, etc. Non-limiting examples of samples include whole blood and blood-derived products such as plasma, serum and buffy coat, urine, feces, cerebrospinal fluid or any other body fluids, tissues, cell cultures, cell suspensions, etc. Other example samples include fluids containing functionalized beads to which a portion of a biologic sample or other particles are attached.

Terms to exemplify orientation may be used herein to refer to relative positions of elements as shown in the figures. It should be understood that the terminology is used for notational convenience and that in actual use the disclosed structures may be oriented different from the orientation shown in the figures. Thus, the terms should not be construed in a limiting manner.

Although figures and examples herein describe microfluidic devices in which fluid channels are shape generally rectangular and the chamber are shapes a rhomboidal or circular, examples are not so limited. For example, the channels and/or chamber may be rectangular, square, oval, circular, rhomboidal, and/or any other shape. The various apparatuses and/or microfluidic device may include more or less numbers of components, such as additional or fewer different reagents and fluid channels.

Various terminology as used in the specification, including the claims, connote a plain meaning unless otherwise indicated. As examples, the specification describes and/or illustrates aspects useful for implementing the claimed disclosure by way of various structure, such as circuitry selected or designed to carry out specific acts or functions, as may be recognized in the figures or the related discussion as depicted by or using terms such as blocks, device, and system, and/or other examples. Certain aspects of these blocks may also be used in combination to exemplify how operational aspects have been designed and/or arranged. Whether alone or in combination with other such blocks or circuitry including discrete circuit elements such as resistors, these above-characterized blocks may be circuits coded by fixed design and/or by configurable circuitry and/or circuit elements for carrying out such operational aspects. In some examples, such a programmable circuitry refers to or includes computer circuits, including memory circuitry for storing and accessing a set of program code to be accessed/executed as instructions and/or configuration data to perform the related operation. Depending on the data-processing application, such instructions and/or data may be for implementation in logic circuitry, with the instructions as may be stored in and accessible from a memory circuit. Such instructions may be stored in and accessible from a memory via a fixed circuitry, a group of configuration code, or instructions characterized by way of object code.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. Therefore, it is intended that this disclosure be limited only by the claims and the equivalents thereof.

1. A microfluidic device, comprising:
a plurality of fluid channels including:
a plurality of dehydrated reagents disposed within the plurality of fluid channels; and
a plurality of fluid blocking regions including blocking material disposed within the plurality of fluid channels;
an unblocking actuator coupled to the plurality of fluid channels proximal to the plurality of fluid blocking regions; and
a fluid ejection device fluidically coupled to the plurality of fluid channels.

2. The microfluidic device of claim 1, wherein the unblocking actuator includes a plurality of control lines and wherein the plurality of fluid blocking regions include the blocking material that is temporarily disposed within the plurality of fluid channels and which are removable via an electrical signal selectively applied thereto via the plurality of control lines, and in response to the selectively applied electrical signal, the flow of fluid is unblocked from an associated fluid channel of the plurality of fluid channels and a respective dehydrated reagent of the plurality of dehydrated reagents is reconstituted in the fluid and directed to the fluid ejection device.

3. The microfluidic device of claim 1, further including a reservoir and a first fluid chamber coupled to the reservoir and the plurality of fluid channels, wherein the reservoir has a layer of breakable material coupled to an inlet of the first fluid chamber, and wherein a force applied to the layer of breakable material causes the reservoir to fluidically couple to the first fluid chamber via the inlet of the first fluid chamber and the plurality of fluid channels are fluidically coupled to an outlet of the first fluid chamber.

4. The microfluidic device of claim 1, wherein the plurality of dehydrated reagents include reagents selected from the group consisting of:
an antibiotic, an enzyme, a nucleotide, an antibody, a metabolic indicator, a detectable label, and combinations thereof.

5. The microfluidic device of claim 1, wherein:
the blocking material is selected from wax, polymer, metal, a magnet, ferrofluid, glass, plastic, and a solid-gas generating material; and
the unblocking actuator includes or forms part of circuitry and structures selected from:
a plurality of control lines to provide heat to the plurality of fluid blocking regions in response to an electrical signal selectively applied thereto;
a pneumatic source to selectively provide a pneumatic signal to the plurality of fluid blocking regions in response to an electrical signal applied thereto;
a plurality of vents with a layer of breakable material disposed proximal to the plurality of fluid blocking regions;
a plurality of magnets disposed proximal to the plurality of fluid blocking regions to provide a magnetic field; and
a plurality of fluid actuators disposed proximal to the plurality of fluid blocking regions.

6. The microfluidic device of claim 1, wherein the fluid ejection device includes a plurality of fluid ejection devices including:
a plurality of second fluid chambers, wherein each of the plurality of second fluid chambers are fluidically coupled to a respective fluid channel of the plurality of fluid channels, and each of the plurality of fluid ejection devices further include a fluid actuator and nozzle to eject fluid therefrom.

7. A non-transitory computer-readable medium storing instructions which, when executed by a processor, cause the processor to:
cause an unblocking actuator coupled to a first fluid blocking region of a plurality of fluid blocking regions to unblock a first fluid channel of a plurality of fluid channels of a microfluidic device, wherein the plurality of fluid channels include:
a plurality of dehydrated reagents disposed within the plurality of fluid channels; and
the plurality of fluid blocking regions including blocking material disposed within the plurality of fluid channels to block flow of the fluid along the plurality of fluid channels;
cause a fluid control actuator to activate flow of fluid to and along the first fluid channel and to a first fluid ejection device fluidically coupled to the first fluid channel, wherein the flow of fluid reconstitutes a first dehydrated reagent of the plurality of dehydrated reagents within the fluid; and
cause the first fluid ejection device to eject a volume of the fluid with the reconstituted first dehydrated reagent from the microfluidic device.

8. The non-transitory computer-readable medium of claim 7, wherein the volume of the fluid is ejected from the microfluidic device to a region of a substrate, and further including instructions that, when executed, cause the processor to:
cause a fluid ejection device of a second microfluidic device to eject a volume of sample fluid to the region of the substrate.

9. The non-transitory computer-readable medium of claim 7, further including instructions that, when executed, cause the processor to:
cause the unblocking actuator coupled to a second fluid blocking region of the plurality of fluid blocking regions to unblock a second fluid channel of the plurality of fluid channels, wherein:
the activation of the flow of fluid causes the fluid to flow along the second fluid channel and to the first fluid ejection device fluidically coupled to the second fluid channel and to reconstitute a second dehydrated reagent of the plurality of dehydrated reagents within the fluid; and
the volume of the fluid ejected includes the reconstituted first dehydrated reagent and the reconstituted second dehydrated reagent.

10. The non-transitory computer-readable medium of claim 7, further including instructions that, when executed, cause the processor to:
cause the unblocking actuator coupled to a second fluid blocking region of the plurality of fluid blocking regions to unblock a second fluid channel of the plurality of fluid channels, wherein the unblocking actuator includes a plurality of control lines and the processor is to apply a first electrical signal to a first control line of the plurality of control lines and a second electrical signal to a second control line of the plurality of control lines to cause application of heat to the first fluid blocking region and the second fluid blocking region;
wherein the activation of the flow of the fluid causes the fluid to flow along the second fluid channel and to a second fluid ejection device fluidically coupled to the second fluid channel, and to reconstitute a second dehydrated reagent of the plurality of reconstituted reagents within the fluid; and
cause the second fluid ejection device to eject a volume of the fluid with the reconstituted second dehydrated reagent from the microfluidic device.

11. The non-transitory computer-readable medium of claim 7, wherein the fluid control actuator forms part of a plunger and the instructions to cause the fluid control actuator to activate the flow of fluid include instructions that, when executed, cause the processor to:
activate the plunger disposed proximal to a reservoir to press on the reservoir; and
in response, break a layer of breakable material of the reservoir and provide fluidic coupling between the reservoir and a first fluid chamber fluidically coupled to the plurality of fluid channels, wherein the activation of the flow of fluid activates fluid flow from the first fluid chamber to each of the plurality of fluid blocking regions, and the remaining plurality of fluid blocking regions block the flow of fluid.

12. A method comprising:
applying heat to a first fluid blocking region of a plurality of fluid blocking regions to unblock a first fluid channel of a plurality of fluid channels of a microfluidic device, wherein the plurality of fluid channels include:
a plurality of dehydrated reagents disposed within the plurality of fluid channels; and
the plurality of fluid blocking regions including blocking material disposed within the plurality of fluid channels between a first fluid chamber of the microfluidic device and the plurality of dehydrated reagents to block flow of the fluid along the plurality of fluid channels;
flowing a fluid from the first fluid chamber to the plurality of fluid blocking regions and along the first fluid channel to a first dehydrated reagent of the plurality of dehydrated reagents disposed within the first fluid channel to reconstitute the first dehydrated reagent within the fluid;
flowing the fluid with the reconstituted first dehydrated reagent from the first fluid channel to a first fluid ejection device of the microfluidic device; and
ejecting a volume of the fluid with the reconstituted first dehydrated reagent from the microfluidic device using the first fluid ejection device.

13. The method of claim 12, further including:
applying heat to a second fluid blocking region of the plurality of fluid blocking regions to unblock a second fluid channel of the plurality of fluid channels;
flowing the fluid from the first fluid chamber and along the second fluid channel to a second dehydrated reagent of the plurality of dehydrated reagents disposed within the second fluid channel to reconstitute the second dehydrated reagent within the fluid;
flowing the fluid with the reconstituted second dehydrated reagent from the second fluid channel to one of the first fluid ejection device and a second fluid ejection device of the microfluidic device; and
ejecting a volume of the fluid with the reconstituted second dehydrated reagent from the microfluidic device using the one of the first fluid ejection device and the second fluid ejection device.

14. The method of claim 12, further including breaking a layer of breakable material of a reservoir containing the fluid to fluidically couple the reservoir to the first fluid chamber and, in response, flowing the fluid from the first fluid chamber to the plurality of fluid blocking regions.

15. The method of claim 12, wherein the volume of the fluid is ejected from the microfluidic device to a region of a substrate, and the method further includes ejecting a volume of sample fluid to the region of the substrate using a fluid ejection device of a second microfluidic device, wherein the microfluidic device and the second microfluidic device are coupled to and disposed within a fluid dispensing device.
